SEQUENCE LISTING (43 sequences)

SEQ ID NO: 1 - DNA, 38 bp, synthetic construct
  source 1..38: mol_type = other DNA
gacctcccagctccatcaaacatctcatcttgatgaaa

SEQ ID NO: 2 - DNA, 20 bp, synthetic construct
  source 1..20: mol_type = other DNA
cagaaggacatttggcctca

SEQ ID NO: 3 - DNA, 19 bp, synthetic construct
  source 1..19: mol_type = other DNA
tgggtgactgatgaaaaag

SEQ ID NO: 4 - DNA, 26 bp, synthetic construct
  source 1..26: mol_type = other DNA
ctcgacaaatgtgagttacagaggga

SEQ ID NO: 5 - DNA, 27 bp, synthetic construct
  source 1..27: mol_type = other DNA
aagatacagatgaagaagaatgaggcg

SEQ ID NO: 6 - DNA, 29 bp, synthetic construct
  source 1..29: mol_type = other DNA
ctagaaaagtgtaagacccgtaatataag

SEQ ID NO: 7 - DNA, 26 bp, synthetic construct
  source 1..26: mol_type = other DNA
ctatgaatgctgtggctattgtcgca

SEQ ID NO: 8 - DNA, 27 bp, synthetic construct
  source 1..27: mol_type = other DNA
gctgatagtagatttgtgatgaccgta

SEQ ID NO: 9 - DNA, 26 bp, synthetic construct
  source 1..26: mol_type = other DNA
ctcagattcactcgacgagggtagta

SEQ ID NO: 10 - DNA, 26 bp, synthetic construct
  source 1..26: mol_type = other DNA
tgaggacaaatatcattytgaggrgc

SEQ ID NO: 11 - DNA, 31 bp, synthetic construct
  source 1..31: mol_type = other DNA
tttccctcagatycattctactagttcagtc

SEQ ID NO: 12 - DNA, 27 bp, synthetic construct
  source 1..27: mol_type = other DNA
aaagaggaggtgaattaagactaaagt

SEQ ID NO: 13 - DNA, 25 bp, synthetic construct
  source 1..25: mol_type = other DNA
atcggaatgggaggtgattcctagg

SEQ ID NO: 14 - DNA, 25 bp, synthetic construct
  source 1..25: mol_type = other DNA
gagtaggagtaaggctcctaggata

SEQ ID NO: 15 - DNA, 28 bp, synthetic construct
  source 1..28: mol_type = other DNA
aatactagggttagtaggagtaggacta

SEQ ID NO: 16 - DNA, 26 bp, synthetic construct
  source 1..26: mol_type = other DNA
ttagaacaagaattagtagcatggcg

SEQ ID NO: 17 - DNA, 38 bp, synthetic construct; identical to SEQ ID NO: 1
  source 1..38: mol_type = other DNA
  modified_base 1: mod_base = OTHER; note = 6-carboxyfluorescein

SEQ ID NO: 18 - DNA, 38 bp, synthetic construct; identical to SEQ ID NO: 1
  source 1..38: mol_type = other DNA
  modified_base 1: mod_base = OTHER; note = 2'-chloro-5'-fluoro-7',8'-benzo-1,4-dichloro-6-carb oxyfluorescein

SEQ ID NO: 19 - DNA, 26 bp, synthetic construct
  source 1..26: mol_type = other DNA
  modified_base 1: mod_base = OTHER; note = 6-carboxyfluorescein
tgaggacaaatatcattytgaggrgc

SEQ ID NO: 20 - DNA, 26 bp, synthetic construct
  source 1..26: mol_type = other DNA
  modified_base 1: mod_base = OTHER; note = 2'-chloro-5'-fluoro-7',8'-benzo-1,4-dichloro-6-carb oxyfluorescein
tgaggacaaatatcattytgaggrgc

SEQ ID NO: 21 - DNA, 97 bp, Felis catus
  source 1..97: mol_type = genomic DNA
aggccaaatgtccttctgaggagcaaccgtaatcactaacctcctgtcagcaattccatacatcgggactgaactagtagaatgaatctgagggggg

SEQ ID NO: 22 - DNA, 98 bp, Felis catus
  source 1..98: mol_type = genomic DNA
gaggccaaatgtccttctgaggagcaaccgtaatcactaacctcctgtcagcaattccatacatcgggactgaactagtagaatgaatctgagggggg

SEQ ID NO: 23 - DNA, 99 bp, Felis catus
  source 1..99: mol_type = genomic DNA
tgaggccaaatgtccttctgaggagcaaccgtaatcactaacctcctgtcagcaattccatacatcgggactgaactagtagaatgaatctgagggggg

SEQ ID NO: 24 - DNA, 143 bp, Canis lupus
  source 1..143: mol_type = genomic DNA
ctcccagcgccgtctaacatctctgcttgatgaaacttcggatccttactaggagtatgcttgattctacagattctaacaggtttattcttagctatgcactatacatcggacacagccacagctttttcatcagtcaccca

SEQ ID NO: 25 - DNA, 144 bp, Canis lupus
  source 1..144: mol_type = genomic DNA
cctcccagcgccgtctaacatctctgcttgatgaaacttcggatccttactaggagtatgcttgattctacagattctaacaggtttattcttagctatgcactatacatcggacacagccacagctttttcatcagtcaccca

SEQ ID NO: 26 - DNA, 145 bp, Canis lupus
  source 1..145: mol_type = genomic DNA
acctcccagcgccgtctaacatctctgcttgatgaaacttcggatccttactaggagtatgcttgattctacagattctaacaggtttattcttagctatgcactatacatcggacacagccacagctttttcatcagtcaccca

SEQ ID NO: 27 - DNA, 151 bp, Capra sp.
  source 1..151: mol_type = genomic DNA
ccaaccccatcaaacatctcatcatgatgaaactttggatccctcctaggaatttgcctaatcttacaaatcctgacaggcctattcctagcaatacactatacatccgacacaataacagcattttcctctgtaactcacatttgtcgag

SEQ ID NO: 28 - DNA, 196 bp, Oryctolagus cuniculus
  source 1..196: mol_type = genomic DNA
atgaggacaaatatcattttggggagcaaccgtaatcactaacctcctatcagcaatcccatatatcggaacaaccttagttgaatgaatctgaggaggattttcagttgataaagccactcttacccgattcttcgcttttcacttcatcttgccatttatcattgcaactttagtcttaattcacctcctcttt

SEQ ID NO: 29 - DNA, 221 bp, Gallus gallus
  source 1..221: mol_type = genomic DNA
ccagccccatccaacatctctgcttgatgaaatttcggctccctattagcagtctgcctcatgacccaaatcctcaccggcctactactagccatgcactacacagcagacacatccctagccttctcctccgtagcccacacttgccggaacgtacaatacggctgactcatccggaatctccacgcaaacggcgcctcattcttcttcatctgtatctt

SEQ ID NO: 30 - DNA, 221 bp, Meleagris gallopavo
  source 1..221: mol_type = genomic DNA
ccaaccccatccaacatctccgcttgatgaaacttcggctccctactagcagtatgcctcatcactcaaatcttaaccggcctcctactagccatacattacactgcagacaccactcttgcattctcttctgtggcctacacatgccgaaacgtacaatacggttgactcctccataacctccatgcgaatggcgcctcattcttcttcatctgcatctt

SEQ ID NO: 31 - DNA, 246 bp, Homo sapiens
  source 1..246: mol_type = genomic DNA
tgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgat

SEQ ID NO: 32 - DNA, 268 bp, Bos taurus
  source 1..268: mol_type = genomic DNA
ccagccccatcaaacatttcatcatgatgaaatttcggttccctcctgggaatctgcctaatcctacaaatcctcacaggcctattcctagcaatacactacacatccgacacaacaacagcattctcctctgttacccatatctgccgagacgtgaactacggctgaatcatccgatacatacacgcaaacggagcttcaatgttttttatctgcttatatatgcacgtaggacgaggcttatattacgggtcttacacttttctag

SEQ ID NO: 33 - DNA, 305 bp, Canis lupus
  source 1..305: mol_type = genomic DNA
tgaggacaaatatcattttgaggagcaactgtaatcactaatcttctctctgccatcccttatatcggaactgacttagtagaatggatctgaggcggcttctcagtggacaaagcaaccctaacacgattctttgcattccatttcatcctccctttcatcatcgcagctctagcaatagtacacctcctatttctacacgaaaccggatccaacaacccttcaggaatcacatcagactcagacaaaattccatttcacccttactacacaatcaaggatatcctaggagccttactcctact

SEQ ID NO: 34 - DNA, 306 bp, Canis lupus
  source 1..306: mol_type = genomic DNA
tgaggacaaatatcattttgaggagcaactgtaatcactaatcttctctctgccatcccttatatcggaactgacttagtagaatggatctgaggcggcttctcagtggacaaagcaaccctaacacgattctttgcattccatttcatcctccctttcatcatcgcagctctagcaatagtacacctcctatttctacacgaaaccggatccaacaacccttcaggaatcacatcagactcagacaaaattccatttcacccttactacacaatcaaggatatcctaggagccttactcctactc

SEQ ID NO: 35 - DNA, 314 bp, Capra sp.
  source 1..314: mol_type = genomic DNA
aggacaaatatcattttgaggggcaacagtcatcactaatcttctttcagcaatcccatatattggcacaaacctagtcgaatgaatctgagggggattctcagtagacaaagccactctcacccgattcttcgccttccactttatcctcccattcatcatcacagccctcgccatagtccacctgcttttcctccacgaaacaggatcgaacaaccccacaggaattccatcagacgcagataaaatcccatttcacccttactacaccattaaagatatcttaggcgccatgctactaattcttgttctaa

SEQ ID NO: 36 - DNA, 315 bp, Capra sp.
  source 1..315: mol_type = genomic DNA
gaggacaaatatcattttgaggggcaacagtcatcactaatcttctttcagcaatcccatatattggcacaaacctagtcgaatgaatctgagggggattctcagtagacaaagccactctcacccgattcttcgccttccactttatcctcccattcatcatcacagccctcgccatagtccacctgcttttcctccacgaaacaggatcgaacaaccccacaggaattccatcagacgcagataaaatcccatttcacccttactacaccattaaagatatcttaggcgccatgctactaattcttgttctaa

SEQ ID NO: 37 - DNA, 316 bp, Capra sp.
  source 1..316: mol_type = genomic DNA
tgaggacaaatatcattttgaggggcaacagtcatcactaatcttctttcagcaatcccatatattggcacaaacctagtcgaatgaatctgagggggattctcagtagacaaagccactctcacccgattcttcgccttccactttatcctcccattcatcatcacagccctcgccatagtccacctgcttttcctccacgaaacaggatcgaacaaccccacaggaattccatcagacgcagataaaatcccatttcacccttactacaccattaaagatatcttaggcgccatgctactaattcttgttctaa

SEQ ID NO: 38 - DNA, 328 bp, Equus asinus
  source 1..328: mol_type = genomic DNA
gaggacaaatatccttctgaggagcaacggtcattacaaacctcctatcagcaatcccctacatcggtactacgctcgtcgaatgaatctgaggtggattctcagtagacaaagccacccttacccgattttttgccttccactttattctaccctttatcatcacagccctggtaatcgtccatctactattcctccacgaaacaggatccaacaacccctcaggaatcccatctgacatagacaaaatcccattccacccgtactacacaattaaagacatcctaggacttctcctcctagtcctactcctactaaccctagtatt

SEQ ID NO: 39 - DNA, 329 bp, Equus asinus
  source 1..329: mol_type = genomic DNA
tgaggacaaatatccttctgaggagcaacggtcattacaaacctcctatcagcaatcccctacatcggtactacgctcgtcgaatgaatctgaggtggattctcagtagacaaagccacccttacccgattttttgccttccactttattctaccctttatcatcacagccctggtaatcgtccatctactattcctccacgaaacaggatccaacaacccctcaggaatcccatctgacatagacaaaatcccattccacccgtactacacaattaaagacatcctaggacttctcctcctagtcctactcctactaaccctagtatt

SEQ ID NO: 40 - DNA, 325 bp, Ovis aries
  source 1..325: mol_type = genomic DNA
ccagctccatcaaatatttcatcatgatgaaactttggctctctcctaggcatttgcttaattttacagattctaacaggcctattcctagcaatacactatacacctgacacaacaacagcattctcctctgtaacccacatttgccgagacgtaaactatggctgaattatccgatatatacacgcaaacggggcatcaatattttttatctgcctatttatgcatgtaggacgaggcctatactatggatcatataccttcctagaaacatgaaacatcggagtaatcctcctatttgcgacaatagccacagcattcatag

SEQ ID NO: 41 - DNA, 359 bp, Felis catus
  source 1..359: mol_type = genomic DNA
cccgccccatctaacatctcagcatgatgaaacttcggctcccttctaggagtctgcctaaccttacaaatcctcaccggcctctttttggccatacactacacatcagacacaataaccgccttttcatcagttacccacatctgtcgcgacgttaattatggctgaatcatccgatatttacacgccaacggagcttctatattctttatctgcctgtacatacatgtaggacggggaatatactacggctcctacaccttctcagagacatgaaacattggaatcatactattatttacagtcatagccacagcttttatgggatacgtcctaccatgaggccaaatgtccttctg

SEQ ID NO: 42 - DNA, 392 bp, Sus scrofa
  source 1..392: mol_type = genomic DNA
ccagccccctcaaacatctcatcatgatgaaacttcggttccctcttaggcatctgcctaatcttgcaaatcctaacaggcctgttcttagcaatacattacacatcagacacaacaacagctttctcatcagttacacacatttgtcgagacgtaaattacggatgagttattcgctatctacatgcaaacggagcatccatattctttatttgcctattcatccacgtaggccgaggtctatactacggatcctatatattcctagaaacatgaaacattggagtagtcctactatttaccgttatagcaacagccttcataggctacgtcctgccctgaggacaaatatcattctgaggagctacggtcatcacaaatctactatcagc

SEQ ID NO: 43 - DNA, 433 bp, Equus caballus
  source 1..433: mol_type = genomic DNA
ccagccccctcaaacatttcatcatgatgaaacttcggctccctcctaggaatctgcctaatcctccaaatcttaacaggcctattcctagccatacactacacatcagacacgacaactgccttctcatccgtcactcacatctgccgagacgttaactacggatgaattattcgctacctccatgccaacggagcatcaatattttttatctgcctcttcattcacgtaggacgcggcctctactacggctcttacacattcctagagacatgaaacattggaatcatcctacttttcacagttatagctacagcattcatgggctatgtcctaccatgaggccaaatatccttttgaggagcaacagtcatcacgaacctcctatcagcaattccctacatcggtactaccctcgtcgagtgaatctgag

(19) United States
(12) Patent Application Publication
(10) Pub. No.: US 2024/0425936 A1
(43) Pub. Date: Dec. 26, 2024

(54) MULTI-SPECIES REFERENCE LADDER AND MULTI-PLEX PCR KIT AND USES THEREOF

(71) Applicants: DeEtta Kay MILLS, Miami, FL (US); Vallarie OSTENSON, Miami, FL (US); Lindsey KAUFMAN, Miami, FL (US); Christina Anne BURNS, Miami, FL (US)

(72) Inventors: DeEtta Kay MILLS, Miami, FL (US); Vallarie OSTENSON, Miami, FL (US); Lindsey KAUFMAN, Miami, FL (US); Christina Anne BURNS, Miami, FL (US)

(73) Assignee: The Florida International University Board of Trustees, Miami, FL (US)

(21) Appl. No.: 18/340,240

(22) Filed: Jun. 23, 2023

(51) Int. Cl. C12Q 1/6888; G01N 21/64

(52) U.S. Cl. CPC C12Q 1/6888; G01N 21/6428; C12Q 1/686; C12Q 2600/16; G01N 2021/6441

(57) ABSTRACT

The subject invention provides multi-species reference ladders, multiplex PCR kits, and methods for multi-species identification using the multi-species reference ladders and multiplex PCR kits. Advantageously, the multi-species reference ladder and multiplex kit allows for a stream-lined analysis process and rapid species identification in a single test.

### SEQUENCE LISTING

The Sequence Listing for this application is labeled “SeqList-21Jun23.xml,” which was created on Jun. 21, 2023, and is 46,183 bytes. The Sequence Listing is incorporated herein by reference in its entirety.

### BACKGROUND

Crimes involving animals have been around for centuries. Dog and cockfighting have been a human blood sport for centuries, with dog fighting having been traced back to Ancient Rome, and cockfighting having been traced back to Ancient Greece. Recently, however, animal cruelty has become a visible, media-driven sensation, but nevertheless is still a horrific crime, as demonstrated by cases like the ones involving Michael Vick in an organized dogfighting ring, and the Bad Newz Kennelz dog fighting investigation.

Animal abuse cases have gained traction in the media in recent years and prompted government officials to increase penalties for animal abuse cases, to amend existing laws, and write more stringent statutes that surround animal abuse cases. Many states are reforming their animal cruelty laws to have greater consequences for crimes against animals, and some states are even broadening their spectrum of animals to be protected under animal cruelty laws. For instance, the state of Florida defines an “animal” to “include every living dumb creature,” the state of New York defines an “animal” as “every living creature except a human being,” and the state of Virginia defines an “animal” as “any non-human vertebrate species.” The new federal law Preventing Animal Cruelty and Torture Act (PACT) was passed in 2019 and “bans intentional crushing, burning, drowning, suffocating, impalement or other serious harm to living non-human mammals, birds, reptiles, or amphibians.”

Forensic science supporting criminal investigations has rapidly developed due to rapid development of scientific and engineering knowledge and analysis methods. For forensic investigations, molecular genetic analysis is performed on forensic science specimens that are various samples collected at crime scenes or large disaster sites. The results obtained from such analysis have a key role in proving a crime or revealing the cause of an accident with regard to the forensic investigation. In particular, detection and analysis of human or non-human DNA in forensic specimens collected at a crime scene may offer primary clues to determining identity of incident subject (e.g., people and/or animals) and reconstruction of a crime scene, thereby contributing to solving the crime.

Currently, forensic identification techniques used in cases involving pets at crime scenes often rely on blood or shed hair analysis as it might relate to the victim or suspect. However, these analyses can be difficult because there are only a few non-human species-specific ‘kits’ available for domestic animals. Most of these (short tandem repeat) STR kits, like the Meowplex used for identifying cats, focus on one species identification. When hair, tissue, or blood are found at a crime scene and no animal is present, a method to identify the unknown species as the source of the evidence is needed before moving to STR analysis.

Analysis of mitochondrial DNA (mtDNA) has become a valuable forensic tool for species identification and authentication. This is possible because cells contain a high number of mtDNA copies, and mtDNA is more resistant to exonuclease digestion. Analysis of mtDNA may become the method of choice for species identification, especially when analysing samples with little or no nuclear DNA. However, even though abundant on a cellular basis, mtDNA quantities may be limited. Thus, judicious use of a sample is important so that sample consumption is minimized and amplification of mtDNA template is optimized.

The standard tool in species identification is mtDNA amplification and sequencing. For species identification, any loci used must show interspecies variation but little intraspecies variation. Loci used are generally the cytochrome b (Cyt-b), cytochrome oxidase (I-III), 12S and 16S rRNA genes. Amplification and then sequencing of the products for species testing is a laborious procedure. Sequence alignments show that there are a limited number of bases that are variable and therefore much of the sequence data are redundant. Many samples collected are mixed with DNA contaminates from other animals. Sample mixtures pose a large problem in that the resulting sequence mixtures are too complicated to decipher.

There are no commercial forensic kits available on the market that can discriminate between domestic species and allow for rapid species determination in collected evidence using a single kit. The current standards in the field of forensics do not allow for discrimination of an unknown species in a stream-lined process without multiple steps, repeated testing, and a process of elimination to discern a species identity.

Thus, there is a need to create a multiple species reference ladder and multiplex protocol that allows for a stream-lined analysis process and rapid species identification, for example, in cases involving animals.

### BRIEF SUMMARY

The subject invention provides reference ladders, kits and methods using the references ladders and kits for multi-species identification and differentiation. The reference ladders, kits and methods of the subject invention provide a quick, robust species identification of unknown sourced evidence collected from, for example, animal fighting rings, illegal slaughter cases, crime scenes involving animal abuse, or food fraud cases.

In certain embodiments, the subject invention provides multi-species reference ladders and accompanying multiplex PCR kits to be used in cases such as animal abuse, blood sports, food fraud, and mixed crime scenes involving domestic animals.

In one embodiment, the subject invention provides a multi-species reference ladder and multiplex PCR kit and methods for multi-species identification using the multi-species reference ladder and multiplex PCR kit. In specific embodiments, the multi-species reference ladder is a mtDNA Cyt-b reference ladder that can be used for species identification using DNA amplicons of Cyt-b gene that can be separated by capillary electrophoresis or gel electrophoresis.

In certain embodiments, the subject invention provides a multiplex kit and/or protocol for PCR that allows for testing two or more species in one reaction instead of having to run multiple experiments to achieve the same result. Such multiplex kit and/or protocol minimizes the reagents being used and time running the analyses.

In specific embodiments, the subject invention provides a stream-lined presumptive identification process of 11 domestic animal species as well as human, which can be utilized in a variety of cases involving animals. Forensic cases involved with animals include, for example, animal abuse cases, sport-fighting of animals, illegal slaughter, and food fraud.

In one embodiment, the subject invention provides a fluorescent multi-species reference ladder comprising two or more (e.g., 2, 3, 4, 5, 6, 7, 8, 9, 10, 11, or 12) DNA fragments of cytochrome b (cyt-b) gene in mitochondrial DNA (mtDNA) from two or more (e.g., 2, 3, 4, 5, 6, 7, 8, 9, 10, 11, or 12) species, each of the two or more DNA fragments having a distinct length.

In specific embodiments, the species are two or more of cat, cow, chicken, turkey, dog, donkey, goat, horse, pig, rabbit, sheep, and human.

In specific embodiments, the fluorescent multi-species reference ladder comprises two or more, three or more, four or more, five or more, six or more, seven or more, eight or more, nine or more, ten or more, eleven or more, twelve or more, thirteen or more of:
97, 98, or 99 bp DNA fragment of cyt-b gene from cat;
143, 144, or 145 bp DNA fragment of cyt-b gene from dog;
155 bp DNA fragment of cyt-b gene from goat;
196 bp DNA fragment of cyt-b gene from rabbit;
226 bp DNA fragment of cyt-b gene from chicken and/or turkey;
248 bp DNA fragment of cyt-b gene from human;
273 bp DNA fragment of cyt-b gene from cow;
305 bp DNA fragment of cyt-b gene from dog;
314, 315, or 316 bp DNA fragment of cyt-b gene from goat;
328 or 329 bp DNA fragment of cyt-b gene from donkey;
329 bp DNA fragment of cyt-b gene from sheep;
363 bp DNA fragment of cyt-b gene from cat;
394 bp DNA fragment of cyt-b gene from pig; and
436 bp DNA fragment of cyt-b gene from horse.

In a specific embodiment, the fluorescent multi-species reference ladder comprises 143, 144, or 145 bp DNA fragment of cyt-b gene from dog; 155 bp DNA fragment of cyt-b gene from goat; 226 bp DNA fragment of cyt-b gene from chicken and/or turkey; 273 bp DNA fragment of cyt-b gene from cow; 329 bp DNA fragment of cyt-b gene from sheep; 363 bp DNA fragment of cyt-b gene from cat; 394 bp DNA fragment of cyt-b gene from pig; and 436 bp DNA fragment of cyt-b gene from horse.

In a specific embodiment, the fluorescent multi-species reference ladder comprises 97, 98, or 99 bp DNA fragment of cyt-b gene from cat; 196 bp DNA fragment of cyt-b gene from rabbit; 248 bp DNA fragment of cyt-b gene from human; 305 bp DNA fragment of cyt-b gene from dog; 314, 315, or 316 bp DNA fragment of cyt-b gene from goat; and 328 or 329 bp DNA fragment of cyt-b gene from donkey.

In specific embodiments, each of the DNA fragments has a fluorescent label, for example, FAM or NED.

In one embodiment, the subject invention provides a kit comprising a fluorescent multi-species reference ladder and a mixture of primers, wherein the primers comprise one or more fluorescent universal forward primers and two or more species-specific reverse primers targeting cyt-b gene in mtDNA.

In a specific embodiment, the one or more fluorescent universal forward primers comprise a sequence of SEQ ID NO: 17 or 18, and/or a sequence of SEQ ID NO:19 or 20.

In specific embodiments, the two or more species-specific reverse primers are selected from:
a sequence comprising SEQ ID NO: 2;
a sequence comprising SEQ ID NO: 3;
a sequence comprising SEQ ID NO: 4;
a sequence comprising SEQ ID NO: 5;
a sequence comprising SEQ ID NO: 6;
a sequence comprising SEQ ID NO: 7;
a sequence comprising SEQ ID NO: 8;
a sequence comprising SEQ ID NO: 9;
a sequence comprising SEQ ID NO: 11;
a sequence comprising SEQ ID NO: 12;
a sequence comprising SEQ ID NO: 13;
a sequence comprising SEQ ID NO: 14;
a sequence comprising SEQ ID NO: 15; and
a sequence comprising SEQ ID NO: 16.

In a preferred embodiment, the kit comprises species-specific reverse primers selected from SEQ ID NOs: 2-9 and/or species-specific reverse primers selected from SEQ ID NOs: 11-16.

In a preferred embodiment, the kit comprises species-specific reverse primers of SEQ ID NOs: 2-9 and/or species-specific reverse primers of SEQ ID NOs: 11-16.

In a specific embodiment, the fluorescent universal forward primer and the two or more species-specific reverse primers are mixed at a ratio of from 1:0.4 to 1:4.

In one embodiment, the subject invention provides a method for species identification in a specimen, e.g., a forensic sample, the method comprising preparing a DNA sample from the specimen, amplifying the DNA sample through multiplex PCR using the kit of the subject invention, and determining the species in the specimen based on the result of the multiplex PCR.

In certain embodiments, the step of amplifying the DNA sample through multiplex PCR comprises mixing the primer mixture with the DNA sample; and subjecting said mixture to multiplex PCR reactions to achieve Cyt-b gene amplicons.

In specific embodiments, the primer mixture comprises: 1) a fluorescent universal forward primer of SEQ ID NO: 17 or 18, and species-specific reverse primers of SEQ ID NOs: 2-9, or 2) a fluorescent universal forward primer of SEQ ID NO: 19 or 20, and species-specific reverse primers of SEQ ID NOs: 11-16.

In certain embodiments, the step of determining the species in the specimen comprising comparing Cyt-b gene amplicons obtained from the multiplex PCR against the fluorescent multi-species reference ladder, wherein the fluorescent multi-species reference ladder comprises: 1) 143 bp DNA fragment for dog; 155 bp DNA fragment for goat; 226 bp DNA fragment for chicken and turkey; 273 bp DNA fragment for cow; 329 bp DNA fragment for sheep; 363 bp DNA fragment for cat; 394 bp DNA fragment for pig; and 436 bp DNA fragment for horse; or 2) 97 bp DNA fragment for cat; 196 bp DNA fragment for rabbit; 248 bp DNA fragment for human; 305 bp DNA fragment for dog; 314 bp DNA fragment for goat; and 328 bp DNA fragment for donkey.

### BRIEF DESCRIPTION OF DRAWINGS

FIG. 1 shows the traditional DNA workflow, including extraction, quantitation and dilution, amplification, and electrophoresis.

FIG. 2 shows the domestic animal reference ladder depicting from left to right: dog (143 bp), goat (155 bp), chicken and turkey (split peak 226 bp), cow (273 bp), sheep (329 bp), cat (363 bp), pig (394 bp) and horse (436 bp). The gray peaks are the size standard.

FIG. 3 shows the gel validation for identification of unknown samples. The gel shows the third round of sampling of the three unknows (A, B and C) in wood (W), cotton (C), and denim (D). The positive controls (PC) were faint, but present (pig and horse). BB: reaction blank; and NC: negative control.

FIG. 4 shows the gel for the unknowns (A, B, and C) exposed to soil(S). The first set (1) was exposed to soil for approximately 1 month, while the other two (2 and 3) sets were exposed for roughly a day or so before extraction. This gel also includes a reaction blank (RBS), a negative control (NC), and two positive controls (PC4 and PC4.1), which were horse and goat, respectively. Goat is expected to have 2 bands (155 bp and 329 bp) under the multiplex conditions and is a distinct identifier for the species.

FIG. 5 shows electropherogram results for the animal reference ladder for multiplex 2. Animal reference ladder was made from combined fluorescently labeled (NED) PCR products. In order from left to right the following black peaks are: cat (97 bp), rabbit (196 bp), human (248 bp), dog (305 bp), goat (314), and donkey (328 bp). The gray peaks are the size standard GSROX500.

FIG. 6 shows gel results for animal reference ladder. Gel is a 3% electrophoresis grade agarose gel. Lane 1 is the 100 bp Ladder. Lane 2 is the animal reference ladder.

### BRIEF DESCRIPTION OF SEQUENCES

SEQ ID NO: 1 is a universal forward primer sequence contemplated for use according to the subject invention.

SEQ ID NOs: 2-9 are reverse primer sequences contemplated for use according to the subject invention.

SEQ ID NO: 10 is a universal forward primer sequence contemplated for use according to the subject invention.

SEQ ID NOs: 11-16 are reverse primer sequences contemplated for use according to the subject invention.

SEQ ID NOs: 17-20 are fluorescent universal forward primer sequences contemplated for use according to the subject invention.

SEQ ID NOs: 21-23 are DNA fragments from cat contemplated for use according to the subject invention.

SEQ ID NOs: 24-26 are DNA fragments from dog contemplated for use according to the subject invention.

SEQ ID NO: 27 is a DNA fragment from goat contemplated for use according to the subject invention.

SEQ ID NO: 28 is a DNA fragment from rabbit contemplated for use according to the subject invention.

SEQ ID NO: 29 is a DNA fragment from chicken contemplated for use according to the subject invention.

SEQ ID NO: 30 is a DNA fragment from turkey contemplated for use according to the subject invention.

SEQ ID NO: 31 is a DNA fragment from human contemplated for use according to the subject invention.

SEQ ID NO: 32 is a DNA fragment from cow contemplated for use according to the subject invention.

SEQ ID NOs: 33-34 are DNA fragments from dog contemplated for use according to the subject invention.

SEQ ID NOs: 35-37 are DNA fragments from goat contemplated for use according to the subject invention.

SEQ ID NOs: 38-39 are DNA fragments from donkey contemplated for use according to the subject invention.

SEQ ID NO: 40 is a DNA fragment from sheep contemplated for use according to the subject invention.

SEQ ID NO: 41 is a DNA fragment from cat contemplated for use according to the subject invention.

SEQ ID NO: 42 is a DNA fragment from pig contemplated for use according to the subject invention.

SEQ ID NO: 43 is a DNA fragment from horse contemplated for use according to the subject invention.

### DETAILED DESCRIPTION

The subject invention provides multi-species reference ladders, multiplex PCR kits, and methods for multi-species identification using the multi-species reference ladders and multiplex PCR kits. Advantageously, the multi-species reference ladder and multiplex kit allows for a stream-lined analysis process and rapid species identification in a single test.

In one embodiment, the multi-species reference ladder of the subject invention for use in species identification comprises DNA amplicons that are generated in multiplex PCR and can be separated by, for example, capillary electrophoresis or gel electrophoresis due to the various sizes of such DNA amplicons. Advantageously, the use of a species-specific reference ladder can eliminate the need for further sequencing as a confirmatory test.

This multi-species reference ladder can be used in a presumptive test when run in conjunction with an unidentified sample from a crime scene. A reference ladder that includes domestic animals can also be used in wildlife crimes to identify or rule out collected unknown samples. Also, often human forensic evidence is comingled with samples originating from typical household pets. The use of an inclusive reference ladder can be beneficial in determining the source of biomaterial collected from the scene and can streamline analysis work. It is crucial to be able to specifically detect target DNA in potentially mixed-species samples.

In one embodiment, the subject invention provides a reference (allelic) ladder for one or more farm animal or domestic animal species selected from, for example, cat, cow, chicken, turkey, dog, donkey, goat, horse, pig, rabbit, and sheep, as well as human.

In one embodiment, the multi-species reference ladder of the subject invention for use in species identification comprises two or more DNA fragments having distinct lengths that can be separated by, for example, capillary electrophoresis or gel electrophoresis.

In specific embodiments, the two or more DNA fragments are amplicons or PCR fragments, having distinguishable lengths, of a gene in mitochondrial DNA (mtDNA) of different species. For species identification, any loci used must show interspecies variation but little intraspecies variation. Loci used are generally the cytochrome b (Cyt-b), cytochrome oxidase (I-III), 12S and 16S rRNA genes.

The mitochondrial cytochrome b gene is used for species identification because it is one of the locations within the mitochondrial genome that has very little intraspecies variation, but significant interspecies variation. Species identification can be based on the length variations of the mt-Cyb gene amplicon.

In a preferred embodiment, the gene in mtDNA is Cyt-b gene. Cyt-b sequence information can be downloaded from NCBI (www.ncbi.nlm.nih.gov/). Cyt-b sequences of cat, cow, dog, donkey, goat, horse, pig, rabbit, sheep, and human have accession numbers of NC_001700, NC_001567, NC_002008, NC_001788, NC_005044, NC_001640, AY337045, NC_001913, NC_001941, and NC_001807, respectively.

In certain embodiments, the subject invention provides a mtDNA Cyt-b reference ladder for species identification, the reference ladder comprising two or more DNA fragments from species selected from, for example, cat, cow, chicken, turkey, dog, donkey, goat, horse, pig, rabbit, sheep, and human, wherein the two or more DNA fragments selected from DNA fragments having lengths of about 97 bp (cat), about 143 bp (dog), about 155 bp (goat), about 196 bp (rabbit), about 226 bp (chicken and turkey), about 248 bp (human), about 273 bp (cow), about 305 bp (dog), about 314 bp (goat), about 328 bp (donkey), about 329 bp (sheep), about 363 bp (cat), about 394 bp (pig), and about 436 bp (horse).

In specific embodiments, the multi-species reference ladder comprising two or more DNA fragments from species selected from, for example, cat, cow, chicken, turkey, dog, donkey, goat, horse, pig, rabbit, sheep, and human, wherein the two or more DNA fragments selected from DNA fragments having lengths of 97 bp (cat), 143 bp (dog), 155 bp (goat), 196 bp (rabbit), 226 bp (chicken and turkey), 248 bp (human), 273 bp (cow), 305 bp (dog), 314 bp (goat), 328 bp (donkey), 329 bp (sheep), 363 bp (cat), 394 bp (pig), and 436 bp (horse).

In specific embodiments, the multi-species reference ladder comprising two or more DNA fragments from species selected from, for example, cat, cow, chicken, turkey, dog, donkey, goat, horse, pig, rabbit, sheep, and human, wherein the two or more DNA fragments selected from DNA fragments having lengths of about 99 bp (cat), about 145 bp (dog), about 155 bp (goat), about 196 bp (rabbit), about 226 bp (chicken and turkey), about 248 bp (human), about 273 bp (cow), about 305 bp (dog), about 315 bp (goat), about 329 bp (donkey), about 329 bp (sheep), about 363 bp (cat), about 394 bp (pig), and about 436 bp (horse).

In specific embodiments, the multi-species reference ladder comprising two or more DNA fragments from species selected from, for example, cat, cow, chicken, turkey, dog, donkey, goat, horse, pig, rabbit, sheep, and human, wherein the two or more DNA fragments selected from DNA fragments having lengths of about 99 bp (cat), about 145 bp (dog), about 151 bp (goat), about 196 bp (rabbit), about 221 bp (chicken and turkey), about 248 bp (human), about 268 bp (cow), about 305 bp (dog), about 315 bp (goat), about 329 bp (donkey), about 325 bp (sheep), about 359 bp (cat), about 392 bp (pig), and about 433 bp (horse).

In a specific embodiment, the multi-species reference ladder comprises one or more DNA fragments each comprising a sequence selected from the following:

| Species | Length | Sequences |
|---|---|---|
| Cat | 97 | 5′-aggccaaat gtccttctga ggagcaaccg taatcactaa cctcctgtca gcaattccat |
| | | acatcgggac tgaactagta gaatgaatct gagggggg-3′ (SEQ ID NO: 21) |
| Cat | 98 | 5′-gaggccaaat gtccttctga ggagcaaccg taatcactaa cctcctgtca gcaattccat |
| | | acatcgggac tgaactagta gaatgaatct gagggggg-3′ (SEQ ID NO: 22) |
| Cat | 99 | 5′-t gaggccaaat gtccttctga ggagcaaccg taatcactaa cctcctgtca gcaattccat |
| | | acatcgggac tgaactagta gaatgaatct gagggggg-3′ (SEQ ID NO: 23) |
| Dog | 143 | 5′-ctcccagc gccgtctaac atctctgctt gatgaaactt cggatcctta ctaggagtat |
| | | gcttgattct acagattcta acaggtttat tcttagctat gcactataca tcggacacag ccacagcttt |
| | | ttcatcagtc accca-3′ (SEQ ID NO: 24) |
| Dog | 144 | 5′-cctcccagc gccgtctaac atctctgctt gatgaaactt cggatcctta ctaggagtat |
| | | gcttgattct acagattcta acaggtttat tcttagctat gcactataca tcggacacag ccacagcttt |
| | | ttcatcagtc accca-3′ (SEQ ID NO: 25) |
| Dog | 145 | 5′-acctcccagc gccgtctaac atctctgctt gatgaaactt cggatcctta ctaggagtat |
| | | gcttgattct acagattcta acaggtttat tcttagctat gcactataca tcggacacag ccacagcttt |
| | | ttcatcagtc accca-3′ (SEQ ID NO: 26) |
| Goat | 151 | 5′-ccaac cccatcaaac atctcatcat gatgaaactt tggatccctc ctaggaattt gcctaatctt |
| | | acaaatcctg acaggcctat tcctagcaat acactataca tccgacacaa taacagcatt |
| | | ttcctctgta actcacattt gtcgag-3′ (SEQ ID NO: 27) |
| Rabbit | 196 | 5′-atgag gacaaatatc attttgggga gcaaccgtaa tcactaacct cctatcagca atcccatata |
| | | tcggaacaac cttagttgaa tgaatctgag gaggattttc agttgataaa gccactctta |
| | | cccgattctt cgcttttcac ttcatcttgc catttatcat tgcaacttta gtcttaattc acctcctctt t-3′ |
| | | (SEQ ID NO: 28) |
| Chicken | 221 | 5′-cc agccccatcc aacatctctg cttgatgaaa tttcggctcc ctattagcag tctgcctcat |
| | | gacccaaatc ctcaccggcc tactactagc catgcactac acagcagaca catccctagc |
| | | cttctcctcc gtagcccaca cttgccggaa cgtacaatac ggctgactca tccggaatct |
| | | ccacgcaaac ggcgcctcat tcttcttcat ctgtatctt-3′ (SEQ ID NO: 29) |
| Turkey | 221 | 5′-ccaa ccccatccaa catctccgct tgatgaaact tcggctccct actagcagta tgcctcatca |
| | | ctcaaatctt aaccggcctc ctactagcca tacattacac tgcagacacc actcttgcat tctcttctgt |
| | | ggcctacaca tgccgaaacg tacaatacgg ttgactcctc cataacctcc atgcgaatgg |
| | | cgcctcattc ttcttcatct gcatctt-3′ (SEQ ID NO: 30) |
| Human | 246 | 5′-tg aggccaaata tcattctgag gggccacagt aattacaaac ttactatccg ccatcccata |
| | | cattgggaca gacctagttc aatgaatctg aggaggctac tcagtagaca gtcccaccct |
| | | cacacgatt tttacctttc acttcatctt gcccttcatt attgcagccc tagcaacact ccacctccta |
| | | ttcttgcacg aaacgggatc aaacaacccc ctaggaatca cctcccattc cgat-3′ (SEQ ID |
| | | NO: 31) |
| COW | 268 | 5′-ccag ccccatcaaa catttcatca tgatgaaatt tcggttccct cctgggaatc tgcctaatcc |
| | | tacaaatcct cacaggccta ttcctagcaa tacactacac atccgacaca acaacagcat |
| | | tctcctctgt tacccatatc tgccgagacg tgaactacgg ctgaatcatc cgatacatac |
| | | acgcaaacgg agcttcaatg ttttttatct gcttatatat gcacgtagga cgaggcttat attacgggtc |
| | | ttacactttt ctag-3′ (SEQ ID NO: 32) |
| Dog | 305 | 5′-tgagga caaatatcat tttgaggagc aactgtaatc actaatcttc tctctgccat cccttatatc |
| | | ggaactgact tagtagaatg gatctgaggc ggcttctcag tggacaaagc aaccctaaca |
| | | cgattctttg cattccattt catcctccct ttcatcatcg cagctctagc aatagtacac ctcctatttc |
| | | tacacgaaac cggatccaac aacccttcag gaatcacatc agactcagac aaaattccat |
| | | ttcaccctta ctacacaatc aaggatatcc taggagcctt actcctact-3′ (SEQ ID NO: 33) |
| Dog | 306 | 5′-tgagga caaatatcat tttgaggagc aactgtaatc actaatcttc tctctgccat cccttatatc |
| | | ggaactgact tagtagaatg gatctgaggc ggcttctcag tggacaaagc aaccctaaca |
| | | cgattctttg cattccattt catcctccct ttcatcatcg cagctctagc aatagtacac ctcctatttc |
| | | tacacgaaac cggatccaac aacccttcag gaatcacatc agactcagac aaaattccat |
| | | ttcaccctta ctacacaatc aaggatatcc taggagcctt actcctactc-3′ (SEQ ID NO: 34) |
| Goat | 314 | 5′-agga caaatatcat tttgaggggc aacagtcatc actaatcttc tttcagcaat cccatatatt |
| | | ggcacaaacc tagtcgaatg aatctgaggg ggattctcag tagacaaagc cactctcacc |
| | | cgattcttcg ccttccactt tatcctccca ttcatcatca cagccctcgc catagtccac ctgcttttcc |
| | | tccacgaaac aggatcgaac aaccccacag gaattccatc agacgcagat aaaatcccat |
| | | ttcaccctta ctacaccatt aaagatatct taggcgccat gctactaatt cttgttctaa-3′ (SEQ ID |
| | | NO: 35) |
| Goat | 315 | 5′-gagga caaatatcat tttgaggggc aacagtcatc actaatctt tttcagcaat cccatatatt |
| | | ggcacaaacc tagtcgaatg aatctgaggg ggattctcag tagacaaagc cactctcacc |
| | | cgattcttcg ccttccactt tatcctccca ttcatcatca cagccctcgc catagtccac ctgcttttcc |
| | | tccacgaaac aggatcgaac aaccccacag gaattccatc agacgcagat aaaatcccat |
| | | ttcaccctta ctacaccatt aaagatatct taggcgccat gctactaatt cttgttctaa-3′ (SEQ ID |
| | | NO: 36) |
| Goat | 316 | 5′-tgagga caaatatcat tttgaggggc aacagtcatc actaatcttc tttcagcaat cccatatatt |
| | | ggcacaaacc tagtcgaatg aatctgaggg ggattctcag tagacaaagc cactctcacc |
| | | cgattcttcg ccttccactt tatcctccca ttcatcatca cagccctcgc catagtccac ctgcttttcc |
| | | tccacgaaac aggatcgaac aaccccacag gaattccatc agacgcagat aaaatcccat |
| | | ttcaccctta ctacaccatt aaagatatct taggcgccat gctactaatt cttgttctaa-3′ (SEQ ID |
| | | NO: 37) |
| Donkey | 328 | 5′-gaggaca aatatccttc tgaggagcaa cggtcattac aaacctccta tcagcaatcc |
| | | cctacatcgg tactacgctc gtcgaatgaa tctgaggtgg attctcagta gacaaagcca |
| | | cccttacccg attttttgcc ttccacttta ttctaccctt tatcatcaca gccctggtaa tcgtccatct |
| | | actattcctc cacgaaacag gatccaacaa cccctcagga atcccatctg acatagacaa |
| | | aatcccattc cacccgtact acacaattaa agacatccta ggacttctcc tcctagtcct |
| | | actcctacta accctagtat t-3′ (SEQ ID NO: 38) |
| Donkey | 329 | 5′-tgaggaca aatatccttc tgaggagcaa cggtcattac aaacctccta tcagcaatcc |
| | | cctacatcgg tactacgctc gtcgaatgaa tctgaggtgg attctcagta gacaaagcca |
| | | cccttacccg attttttgcc ttccacttta ttctaccctt tatcatcaca gccctggtaa tcgtccatct |
| | | actattcctc cacgaaacag gatccaacaa cccctcagga atcccatctg acatagacaa |
| | | aatcccattc cacccgtact acacaattaa agacatccta ggacttctcc tcctagtcct |
| | | actcctacta accctagtat t-3′ (SEQ ID NO: 39) |
| Sheep | 325 | 5′-ccagctcca tcaaatattt catcatgatg aaactttggc tctctcctag gcatttgctt aattttacag |
| | | attctaacag gcctattcct agcaatacac tatacacctg acacaacaac agcattctcc |
| | | tctgtaaccc acatttgccg agacgtaaac tatggctgaa ttatccgata tatacacgca |
| | | aacggggcat caatattttt tatctgccta tttatgcatg taggacgagg cctatactat ggatcatata |
| | | ccttcctaga aacatgaaac atcggagtaa tcctcctatt tgcgacaata gccacagcat tcatag- |
| | | 3′ (SEQ ID NO: 40) |
| Cat | 359 | 5′-cccgccccat ctaacatctc agcatgatga aacttcggct cccttctagg agtctgccta |
| | | accttacaaa tcctcaccgg cctctttttg gccatacact acacatcaga cacaataacc |
| | | gccttttcat cagttaccca catctgtcgc gacgttaatt atggctgaat catccgatat ttacacgcca |
| | | acggagcttc tatattcttt atctgcctgt acatacatgt aggacgggga atatactacg |
| | | gctcctacac cttctcagag acatgaaaca ttggaatcat actattattt acagtcatag ccacagcttt |
| | | tatgggatac gtcctaccat gaggccaaat gtccttctg-3′ (SEQ ID NO: 41) |
| Pig | 392 | 5′-ccagcc ccctcaaaca tctcatcatg atgaaacttc ggttccctct taggcatctg cctaatcttg |
| | | caaatcctaa caggcctgtt cttagcaata cattacacat cagacacaac aacagctttc |
| | | tcatcagtta cacacatttg tcgagacgta aattacggat gagttattcg ctatctacat |
| | | gcaaacggag catccatatt ctttatttgc ctattcatcc acgtaggccg aggtctatac |
| | | tacggatcct atatattcct agaaacatga aacattggag tagtcctact atttaccgtt |
| | | atagcaacag ccttcatagg ctacgtcctg ccctgaggac aaatatcatt ctgaggagct |
| | | acggtcatca caaatctact atcagc-3′ (SEQ ID NO: 42) |
| Horse | 433 | 5′-ccagccccct caaacatttc atcatgatga aacttcggct ccctcctagg aatctgccta |
| | | atcctccaaa tcttaacagg cctattccta gccatacact acacatcaga cacgacaact |
| | | gccttctcat ccgtcactca catctgccga gacgttaact acggatgaat tattcgctac |
| | | ctccatgcca acggagcatc aatatttttt atctgcctct tcattcacgt aggacgcggc |
| | | ctctactacg gctcttacac attcctagag acatgaaaca ttggaatcat cctacttttc acagttatag |
| | | ctacagcatt catgggctat gtcctaccat gaggccaaat atccttttga ggagcaacag |
| | | tcatcacgaa cctcctatca gcaattccct acatcggtac taccctcgtc gagtgaatct gag-3′ |
| | | (SEQ ID NO: 43) |

In certain embodiments, the multi-species reference ladder comprises two or more DNA fragments from species selected from, for example, cat, cow, chicken, turkey, dog, goat, horse, pig, and sheep, wherein the two or more DNA fragments selected from DNA fragments having lengths of about 143 bp (dog), about 155 bp (goat), about 226 bp (chicken and turkey), about 273 bp (cow), about 329 bp (sheep), about 363 bp (cat), about 394 bp (pig), and about 436 bp (horse).

In certain embodiments, the multi-species reference ladder comprises two or more DNA fragments from species selected from, for example, cat, cow, chicken, turkey, dog, goat, horse, pig, and sheep, wherein the two or more DNA fragments selected from DNA fragments having lengths of about 145 bp (dog), about 155 bp (goat), about 226 bp (chicken and turkey), about 273 bp (cow), about 329 bp (sheep), about 363 bp (cat), about 394 bp (pig), and about 436 bp (horse).

In certain embodiments, the multi-species reference ladder comprises two or more DNA fragments from species selected from, for example, cat, dog, donkey, goat, rabbit, and human, wherein the two or more DNA fragments selected from DNA fragments having lengths of about 97 bp (cat), about 196 bp (rabbit), about 248 bp (human), about 305 bp (dog), about 314 bp (goat), and about 328 bp (donkey).

In certain embodiments, the multi-species reference ladder comprises two or more DNA fragments from species selected from, for example, cat, dog, donkey, goat, rabbit, and human, wherein the two or more DNA fragments selected from DNA fragments having lengths of about 99 bp (cat), about 196 bp (rabbit), about 248 bp (human), about 305 bp (dog), about 315 bp (goat), and about 329 bp (donkey).

In certain embodiments, the subject invention provides a reference ladder comprising one or more, two or more, three or more, four or more, five or more, six or more, seven or more, eight or more, nine or more, ten or more, or eleven or more DNA fragments from species selected from, for example, cat, cow, chicken, turkey, dog, donkey, goat, horse, pig, rabbit, sheep, and human, wherein the one or more, two or more, three or more, four or more, five or more, six or more, seven or more, eight or more, nine or more, ten or more, or eleven or more DNA fragments selected from DNA fragments having lengths of about 97 bp (cat), about 143 bp (dog), about 155 bp (goat), about 196 bp (rabbit), about 226 bp (chicken and turkey), about 248 bp (human), about 273 bp (cow), about 305 bp (dog), about 314 bp (goat), about 328 bp (donkey), about 329 bp (sheep), about 363 bp (cat), about 394 bp (pig), and about 436 bp (horse).

In certain embodiments, the reference ladder comprises one or more DNA fragments from species selected from, for example, cat, cow, chicken, turkey, dog, donkey, goat, horse, pig, rabbit, and human, wherein the one or more DNA fragments selected from DNA fragments having lengths of about 97 bp (cat), about 143 bp (dog), about 155 bp (goat), about 196 bp (rabbit), about 226 bp (chicken and turkey), about 248 bp (human), about 273 bp (cow), about 305 bp (dog), about 314 bp (goat), about 328 bp (donkey), about 363 bp (cat), about 394 bp (pig), and about 436 bp (horse).

In certain embodiments, the reference ladder comprises one or more DNA fragments from species selected from, for example, cat, cow, chicken, turkey, dog, donkey, goat, horse, pig, rabbit, and human, wherein the one or more DNA fragments selected from DNA fragments having lengths of about 99 bp (cat), about 145 bp (dog), about 155 bp (goat), about 196 bp (rabbit), about 226 bp (chicken and turkey), about 248 bp (human), about 273 bp (cow), about 305 bp (dog), about 315 bp (goat), about 329 bp (donkey), about 363 bp (cat), about 394 bp (pig), and about 436 bp (horse).

In certain embodiments, the reference ladder comprises one or more DNA fragments from species selected from, for example, cat, cow, chicken, turkey, dog, goat, horse, pig, rabbit, sheep, and human, wherein the one or more DNA fragments selected from DNA fragments having lengths of about 97 bp (cat), about 143 bp (dog), about 155 bp (goat), about 196 bp (rabbit), about 226 bp (chicken and turkey), about 248 bp (human), about 273 bp (cow), about 305 bp (dog), about 314 bp (goat), about 329 bp (sheep), about 363 bp (cat), about 394 bp (pig), and about 436 bp (horse).

In certain embodiments, the reference ladder comprises one or more DNA fragments from species selected from, for example, cat, cow, chicken, turkey, dog, goat, horse, pig, rabbit, sheep, and human, wherein the one or more DNA fragments selected from DNA fragments having lengths of about 99 bp (cat), about 145 bp (dog), about 155 bp (goat), about 196 bp (rabbit), about 226 bp (chicken and turkey), about 248 bp (human), about 273 bp (cow), about 305 bp (dog), about 315 bp (goat), about 329 bp (sheep), about 363 bp (cat), about 394 bp (pig), and about 436 bp (horse).

In certain embodiments, the reference ladder comprises one or more DNA fragments from species selected from, for example, cat, cow, chicken, turkey, dog, donkey, goat, horse, pig, rabbit, and human, wherein the one or more DNA fragments selected from DNA fragments having lengths of about 143 bp (dog), about 155 bp (goat), about 196 bp (rabbit), about 226 bp (chicken and turkey), about 248 bp (human), about 273 bp (cow), about 305 bp (dog), about 328 bp (donkey), about 363 bp (cat), about 394 bp (pig), and about 436 bp (horse).

In certain embodiments, the reference ladder comprises one or more DNA fragments from species selected from, for example, cat, cow, chicken, turkey, dog, donkey, goat, horse, pig, rabbit, and human, wherein the one or more DNA fragments selected from DNA fragments having lengths of about 145 bp (dog), about 155 bp (goat), about 196 bp (rabbit), about 226 bp (chicken and turkey), about 248 bp (human), about 273 bp (cow), about 305 bp (dog), about 329 bp (donkey), about 363 bp (cat), about 394 bp (pig), and about 436 bp (horse).

In certain embodiments, the reference ladder comprises one or more DNA fragments from species selected from, for example, cat, cow, chicken, turkey, dog, goat, horse, pig, rabbit, sheep, and human, wherein the one or more DNA fragments selected from DNA fragments having lengths of about 97 bp (cat), about 143 bp (dog), about 155 bp (goat), about 196 bp (rabbit), about 226 bp (chicken and turkey), about 248 bp (human), about 273 bp (cow), about 305 bp (dog), about 329 bp (sheep), about 394 bp (pig), and about 436 bp (horse).

In certain embodiments, the reference ladder comprises one or more DNA fragments from species selected from, for example, cat, cow, chicken, turkey, dog, goat, horse, pig, rabbit, sheep, and human, wherein the one or more DNA fragments selected from DNA fragments having lengths of about 99 bp (cat), about 145 bp (dog), about 155 bp (goat), about 196 bp (rabbit), about 226 bp (chicken and turkey), about 248 bp (human), about 273 bp (cow), about 305 bp (dog), about 329 bp (sheep), about 394 bp (pig), and about 436 bp (horse).

In specific embodiments, the DNA fragment from cat of the reference ladder comprises a sequence of SEQ ID NO: 21, 22, 23 or 41; the DNA fragment from dog of the reference ladder comprises a sequence of SEQ ID NO: 24, 25, 26, 33 or 34; the DNA fragment from goat of the reference ladder comprises a sequence of SEQ ID NO: 27, 35, 36 or 37; the DNA fragment from rabbit of the reference ladder comprises a sequence of SEQ ID NO: 28; the DNA fragment from chicken of the reference ladder comprises a sequence of SEQ ID NO: 29; the DNA fragment from turkey of the reference ladder comprises a sequence of SEQ ID NO: 30; the DNA fragment from human of the reference ladder comprises a sequence of SEQ ID NO: 31; the DNA fragment from cow of the reference ladder comprises a sequence of SEQ ID NO: 32; the DNA fragment from donkey of the reference ladder comprises a sequence of SEQ ID NO: 38 or 39; the DNA fragment from sheep of the reference ladder comprises a sequence of SEQ ID NO: 40; the DNA fragment from pig of the reference ladder comprises a sequence of SEQ ID NO: 42; and the DNA fragment from horse of the reference ladder comprises a sequence of SEQ ID NO: 43.

In a specific embodiment, the 99 bp DNA fragment from cat of the reference ladder comprises a sequence of SEQ ID NO: 21, 22, or 23; the 98 bp DNA fragment from cat of the reference ladder comprises a sequence of SEQ ID NO: 21, or 22; and the 97 bp DNA fragment from cat of the reference ladder comprises a sequence of SEQ ID NO: 21.

In a specific embodiment, the 145 bp DNA fragment from dog of the reference ladder comprises a sequence of SEQ ID NO: 25, 26, or 24; the 144 bp DNA fragment from dog of the reference ladder comprises a sequence of SEQ ID NO: 25, or 24; and the 143 bp DNA fragment from dog of the reference ladder comprises a sequence of SEQ ID NO: 24.

In a specific embodiment, the 316 bp DNA fragment from goat of the reference ladder comprises a sequence of SEQ ID NO: 35, 36, or 37; the 315 bp DNA fragment from cat of the reference ladder comprises a sequence of SEQ ID NO: 35, or 36; and the 314 bp DNA fragment from cat of the reference ladder comprises a sequence of SEQ ID NO: 35.

In a specific embodiment, the 329 bp DNA fragment from donkey of the reference ladder comprises a sequence of SEQ ID NO: 38 or 39; and the 328 bp DNA fragment from donkey of the reference ladder comprises a sequence of SEQ ID NO: 38.

In some embodiments, the DNA fragments of the reference ladder are fluorescently labelled. Because there is currently no fluorescent reference DNA ladder for multiple animal species that can be used as a presumptive or confirmatory test. The creation of such ladder would ease workflow, provide clarity to analysis, and offer additional methods for meat tempering casework or unknown sourced crime scene samples such as from animal fighting cases. The fluorescent reference ladder is created using a method driven approach to bridge the gap in current technology provides streamlined analysis of unknown multi-species samples. It is vital to maximize the amount of information gleaned from each analytical forensic test, and the creation of this reference ladder would provide a panel of information in one test.

In certain embodiments, the DNA fragments of the reference ladder comprise a fluorescent label, for example, fluorescein, TAMRA, rhodamine, Texas Red, Alexa Fluor (e.g., AlexaFluor 488, AlexaFluor 532, AlexaFluor 546, AlexaFluor 594, AlexaFluor 633 and AlexaFluor 647), cyanine dye (e.g., Cy7, Cy7.5, Cy5, Cy5.5 and Cy3), Tye dye (e.g., TYE 563, TYE 665, TYE 705), atto dye (e.g., Atto 594 and Atto 633), Hexachlorofluorescein, FAM (6-carboxyfluroescein), BODIPY FL, OliGreen, 40,6-diamidino-2-phenylindol (DAPI), Hoechst 33,258, malachite green (MG), or FITC.

In certain embodiments, the reference ladder can be generated by a multiplex PCR using a universal forward primer and species-specific reverse primers. Species-specific primers were designed to only react with the species for which they were designed and such that the size of the amplified fragment could not be confused with any other fragments.

In certain embodiments, the PCR primers were created to target specific regions of the Cyt-b gene in mtDNA. A universal forward primer and species-specific reverse primers were used to differentiate species by amplicon length differences. Amplicon lengths for individual species are variable enough to give clear results. For instance, the fragment achieved when amplified chicken is 226 bp, and the fragment achieved when amplified horse is 437 bp.

In one embodiment, the subject invention provides a multiplex PCR kit comprising a fluorescently labeled universal forward primer that is common to all species, and species-specific reverse primers such that distinct individual amplicons are achieved for presumptive species identification.

In one embodiment, the subject invention provides a multiplex PCR kit for species identification comprising one or more pairs of PCR primers, wherein the PCR primers are created to target specific regions of the Cyt-b gene in the mtDNA and produce an amplification product at cyt-b loci. In one embodiment, the pair of PCR primers include a universal forward primer and a species-specific reverse primer. The one or more pairs of primers are used to differentiate species by amplicon length differences.

In a specific embodiment, the universal forward primer comprises a sequence of SEQ ID NO: 1 (5′-GACCTCCCAGCTCCATCAAACATCTCATCTTGATGAAA-3′), SEQ ID NO: 10 (5′-TGAGGACAAATATCATTYTGAGGRGC-3′), SEQ ID NO: 17 (5′-FAM-GACCTCCCAGCTCCATCAAACATCTCATCTTGATGAAA-3′), SEQ ID NO: 18 (5′-NED-GACCTCCCAGCTCCATCAAACATCTCATCTTGATGAAA-3′), SEQ ID NO: 19 (5′-FAM-TGAGGACAAATATCATTYTGAGGRGC-3′), or SEQ ID NO: 20 (5′-NED-TGAGGACAAATATCATTYTGAGGRGC-3′). In a specific embodiment, the species-specific reverse primer comprises a sequence selected from SEQ ID NOs: 2-9, and 11-16.

In specific embodiments, the multiplex PCR kit comprises one or more, two or more, three or more, four or more, five or more, six or more, seven or more, eight or more, nine or more, ten or more, eleven or more, twelve or more, or thirteen or more of:
a pair of primers of SEQ ID NOs: 1 and 2;
a pair of primers of SEQ ID NOs: 1 and 3;
a pair of primers of SEQ ID NOs: 1 and 4;
a pair of primers of SEQ ID NOs: 1 and 5;
a pair of primers of SEQ ID NOs: 1 and 6;
a pair of primers of SEQ ID NOs: 1 and 7;
a pair of primers of SEQ ID NOs: 1 and 8;
a pair of primers of SEQ ID NOs: 1 and 9;
a pair of primers of SEQ ID NOs: 10 and 11;
a pair of primers of SEQ ID NOs: 10 and 12;
a pair of primers of SEQ ID NOs: 10 and 13;
a pair of primers of SEQ ID NOs: 10 and 14;
a pair of primers of SEQ ID NOs: 10 and 15; and
a pair of primers of SEQ ID NOs: 10 and 16;

In specific embodiments, the multiplex PCR kit comprises one or more, two or more, three or more, four or more, five or more, six or more, seven or more, eight or more, nine or more, ten or more, eleven or more, twelve or more, or thirteen or more of:
a pair of primers of SEQ ID NOs: 17 and 2;
a pair of primers of SEQ ID NOs: 17 and 3;
a pair of primers of SEQ ID NOs: 17 and 4;
a pair of primers of SEQ ID NOs: 17 and 5;
a pair of primers of SEQ ID NOs: 17 and 6;
a pair of primers of SEQ ID NOs: 17 and 7;
a pair of primers of SEQ ID NOs: 17 and 8;
a pair of primers of SEQ ID NOs: 17 and 9;
a pair of primers of SEQ ID NOs: 19 and 11;
a pair of primers of SEQ ID NOs: 19 and 12;
a pair of primers of SEQ ID NOs: 19 and 13;
a pair of primers of SEQ ID NOs: 19 and 14;
a pair of primers of SEQ ID NOs: 19 and 15; and
a pair of primers of SEQ ID NOs: 19 and 16;

In specific embodiments, the multiplex PCR kit comprises one or more, two or more, three or more, four or more, five or more, six or more, seven or more, eight or more, nine or more, ten or more, eleven or more, twelve or more, or thirteen or more of:
a pair of primers of SEQ ID NOs: 18 and 2;
a pair of primers of SEQ ID NOs: 18 and 3;
a pair of primers of SEQ ID NOs: 18 and 4;
a pair of primers of SEQ ID NOs: 18 and 5;
a pair of primers of SEQ ID NOs: 18 and 6;
a pair of primers of SEQ ID NOs: 18 and 7;
a pair of primers of SEQ ID NOs: 18 and 8;
a pair of primers of SEQ ID NOs: 18 and 9;
a pair of primers of SEQ ID NOs: 20 and 11;
a pair of primers of SEQ ID NOs: 20 and 12;
a pair of primers of SEQ ID NOs: 20 and 13;
a pair of primers of SEQ ID NOs: 20 and 14;
a pair of primers of SEQ ID NOs: 20 and 15; and
a pair of primers of SEQ ID NOs: 20 and 16;

In one embodiment, the subject invention provides a multiplex PCR kit for species identification in a forensic case comprises a multi-species reference ladder of the subject invention. The same primer sets were used to create the species reference ladder. In certain embodiments, forensic cases that are directly involved with animals include, for example, animal abuse cases, sport-fighting of animals, illegal slaughter, and food fraud.

Advantageously, the reference ladder and the multiplex kit provide a quick, robust species identification of unknown sourced evidence collected from, for example, animal fighting rings, illegal slaughter cases, crime scenes involving animal abuse, or food fraud cases.

In certain embodiments, the subject invention provides a multiplex PCR kit comprising a multi-species reference ladder of the subject invention and one or more primer pairs of the subject invention. The multiplex PCR kit can be used to quickly distinguish between common domestic pets in cases involving animals and allows uses to distinguish between multiple animals using a single kit.

In certain embodiments, the multiplex PCR kit comprises a multi-species reference ladder of the subject invention and two or more primer pairs of the subject invention. In certain embodiments, the multiplex PCR kit comprises a multi-species reference ladder of the subject invention and three or more primer pairs of the subject invention. In certain embodiments, the multiplex PCR kit comprises a multi-species reference ladder of the subject invention and four or more primer pairs of the subject invention. In certain embodiments, the multiplex PCR kit comprises a multi-species reference ladder of the subject invention and five or more primer pairs of the subject invention. In certain embodiments, the multiplex PCR kit comprises a multi-species reference ladder of the subject invention and six or more primer pairs of the subject invention. In certain embodiments, the multiplex PCR kit comprises a multi-species reference ladder of the subject invention and seven or more primer pairs of the subject invention. In certain embodiments, the multiplex PCR kit comprises a multi-species reference ladder of the subject invention and eight or more primer pairs of the subject invention. In certain embodiments, the multiplex PCR kit comprises a multi-species reference ladder of the subject invention and nine or more primer pairs of the subject invention. In certain embodiments, the multiplex PCR kit comprises a multi-species reference ladder of the subject invention and ten or more primer pairs of the subject invention. In certain embodiments, the multiplex PCR kit comprises a multi-species reference ladder of the subject invention and eleven or more primer pairs of the subject invention. In certain embodiments, the multiplex PCR kit comprises a multi-species reference ladder of the subject invention and twelve or more primer pairs of the subject invention. In certain embodiments, the multiplex PCR kit comprises a multi-species reference ladder of the subject invention and thirteen or more primer pairs of the subject invention.

In certain embodiments, the subject invention provides a multiplex PCR kit comprising a multi-species reference ladder of the subject invention, and a mixture of one or more universal forward primer and one or more species-specific reverse primers.

In certain embodiments, the multiplex PCR kit comprises a multi-species reference ladder of the subject invention, and a mixture of one or more universal forward primer and two or more species-specific reverse primers. In certain embodiments, the multiplex PCR kit comprises a multi-species reference ladder of the subject invention, and a mixture of one or more universal forward primer and three or more species-specific reverse primers. In certain embodiments, the multiplex PCR kit comprises a multi-species reference ladder of the subject invention, and a mixture of one or more universal forward primer and four or more species-specific reverse primers. In certain embodiments, the multiplex PCR kit comprises a multi-species reference ladder of the subject invention, and a mixture of one or more universal forward primer and five or more species-specific reverse primers. In certain embodiments, the multiplex PCR kit comprises a multi-species reference ladder of the subject invention, and a mixture of one or more universal forward primer and five or more species-specific reverse primers. In certain embodiments, the multiplex PCR kit comprises a multi-species reference ladder of the subject invention, and a mixture of one or more universal forward primer and six or more species-specific reverse primers.

In specific embodiments, the multiplex PCR kit comprises a multi-species reference ladder that comprises two or more DNA fragments selected from for example, cat, cow, chicken, turkey, dog, donkey, goat, horse, pig, rabbit, sheep, and human, wherein each of the two or more DNA fragments comprises a sequence selected from SEQ ID NOs: 21-43.

In specific embodiments, the fluorescent universal forward primer comprises a sequence of SEQ ID NO: 17, 18, 19 or 20. In specific embodiments, the species-specific reverse primers are selected from:
a sequence comprising SEQ ID NO: 2;
a sequence comprising SEQ ID NO: 3;
a sequence comprising SEQ ID NO: 4;
a sequence comprising SEQ ID NO: 5;
a sequence comprising SEQ ID NO: 6;
a sequence comprising SEQ ID NO: 7;
a sequence comprising SEQ ID NO: 8
a sequence comprising SEQ ID NO: 9;
a sequence comprising SEQ ID NO: 11;
a sequence comprising SEQ ID NO: 12;
a sequence comprising SEQ ID NO: 13;
a sequence comprising SEQ ID NO: 14;
a sequence comprising SEQ ID NO: 15; and
a sequence comprising SEQ ID NO: 16.

In specific embodiments, the multiplex PCR kit comprises a multi-species reference ladder of the subject invention, and a mixture of a universal forward primer of SEQ ID NO: 17 or 18 and species-specific reverse primers of SEQ ID NOs: 2-9.

In specific embodiments, the multiplex PCR kit comprises a multi-species reference ladder of the subject invention, and a mixture of a universal forward primer of SEQ ID NO: 19 or 20 and species-specific reverse primers of SEQ ID NOs: 11-16.

In some embodiments, the fluorescent universal forward primer and the species-specific reverse primers are mixed at a ratio of from 1:0.4 to 1:4, 1:0.5 to 1:4, from 1:0.6 to 1:4, from 1:0.7 to 1:4, from 1:0.8 to 1:4, from 1:0.9 to 1:4, from 1:1 to 1:4, from 1:1.2 to 1:4, from 1:1.5 to 1:4, from 1:1.7 to 1:4, from 1:2 to 1:4, or from 1:2.5 to 1:4.

In a specific embodiment, the fluorescent universal forward primer and the species-specific reverse primers: SEQ ID NOs: 2-9 are mixed at a ratio of 1:0.33:0.67:0.67:0.67:0.33:0.2:0.2:0.8.

In some embodiments, the multiplex PCR kit of the subject invention may further comprise DNA polymerases, dNTPs, PCR buffer and/or a standard ladder.

In one embodiment, the subject invention provides a method of using the multiplex PCR kit for species identification by targeting the Cyt-b gene in mitochondrial DNA. In specific embodiments, the species include, for example, cat, dog, horse, sheep, goat, pig, rabbit, donkey, cow, chicken, and turkey, as well as human.

In one embodiment, the subject invention provides a stream-lined presumptive identification process of one or more domestic animal species as well as human that can be utilized in a variety of cases involving animals. Advantageously, the method of subject invention optimizes the process of domestic species identification and allows for presumptive results to be achieved where current standards call for repeat testing, and a process of trial and error.

In one embodiment, the subject invention provides a method of using the multiplex PCR kit of the subject invention for identify species in a sample, e.g., a specimen collected for forensic investigation. In one embodiment, the method for identifying one or more species in a specimen comprises:
preparing a DNA sample from the specimen, preferably, the specimen can be a sample or forensic sample corrected for forensic investigation;
amplifying the DNA sample through multiplex PCR using the multiplex PCR kit of the subject invention; and
determining the species in the specimen based on the result of the multiplex PCR.

In one embodiment, amplifying the DNA sample through multiplex PCR using the multiplex PCR kit comprises mixing a primer mixture of a fluorescent universal forward primer and species-specific reverse primers with the DNA sample, and subjecting such mixture of primers and DNA sample to multiplex PCR reactions to achieve Cyt-b gene amplicons.

In certain embodiments, the primers are mixed together in specific concentrations to make a primer mix that yields amplicons of even intensity and amplification is achieved using PCR with very specific thermocycler conditions.

The same primer sets were used to create the species reference ladder that were in the multiplex reactions. DNA from each individual species is run against all other species to ensure no cross-amplification and that distinct individual species identification could be achieved. When the amplicon is compared against the reference ladder, the unknown species can be properly identified.

In specific embodiments, the primer mixture comprises a fluorescent universal forward primer selected from a sequence of SEQ ID NO: 17 or 18, and a sequence of SEQ ID NO: 19 or 20; and species-specific reverse primers selected from one or more sequences of SEQ ID NOs: 2-9; and one or more sequences of SEQ ID NOs: 11-16.

In a specific embodiment, the primer mixture comprises a fluorescent universal forward primer of SEQ ID NO: 17 or 18, and species-specific reverse primers of SEQ ID NOs: 2-9.

In a specific embodiment, the primer mixture comprises a fluorescent universal forward primer of SEQ ID NO: 19 or 20, and species-specific reverse primers of SEQ ID NOs: 11-16.

In one embodiment, determining the species in the specimen comprises determining the Cyt-b gene amplicon obtained from the multiplex PCR reactions; comparing the amplicon to the multi-species reference ladder; and determining the species in the specimen.

In specific embodiments, the fluorescent multi-species reference ladder comprises 97, 98, or 99 bp DNA fragment for cat; 143, 144 or 145 bp DNA fragment for dog; 155 bp DNA fragment for goat; 196 bp DNA fragment for rabbit; 226 bp DNA fragment for chicken and/or turkey; 248 bp DNA fragment for human; 273 bp DNA fragment for cow; 305 bp DNA fragment for dog; 314, 315, or 316 bp DNA fragment for goat; 328 or 329 bp DNA fragment for donkey; 329 bp DNA fragment for sheep; 363 bp DNA fragment for cat; 394 bp DNA fragment for pig; 436 bp DNA fragment for horse; or any combinations thereof.

In specific embodiments, the fluorescent multi-species reference ladder comprises 143, 144, or 145 bp DNA fragment for dog; 155 bp DNA fragment for goat; 226 bp DNA fragment for chicken and/or turkey; 273 bp DNA fragment for cow; 329 bp DNA fragment for sheep; 363 bp DNA fragment for cat; 394 bp DNA fragment for pig; and 436 bp DNA fragment for horse.

In specific embodiments, the fluorescent multi-species reference ladder comprises 97 bp DNA fragment for cat; 196 bp DNA fragment for rabbit; 248 bp DNA fragment for human; 305 bp DNA fragment for dog; 314 bp DNA fragment for goat; and 328 bp DNA fragment for donkey.

In one embodiment, the subject invention provides a method for identifying species in a sample, e.g., a specimen collected for forensic investigation, the method comprising:
extracting DNA from the sample and quantifying the extracted DNA,
amplifying a gene using the multiplex PCR kit of the subject invention,
collecting amplicons of the gene obtained from the multiplex PCR reactions,
comparing the amplicons against the reference ladder of the subject invention, and
identifying the species in the sample.

Advantageously, the multiplex allows for testing, for example, 12 individual species in two reactions instead of having to run multiple experiments to get the result. It minimizes the reagents being used and time running the analyses. The creation of the reference ladder that is included in the kit does not exist on the market for multiple species identification. The equipment required to run each analysis is standard in any forensic lab, so no extra equipment is required. Unlabeled primers along with gel electrophoresis equipment can be used with this methodology which will significantly cut down on expenses. The kit also has the ability to differentiate between humans and 11 domestic animal species, which allows investigators to quickly include or exclude humans as suspect.

In specific embodiments, the step of comparing the amplicons against the reference ladder of the subject invention comprises separating DNA amplicons by, for example, capillary electrophoresis or gel electrophoresis for species identification using the multi-species reference ladder.

In specific embodiments, the subject invention provides two reference ladders, combined to run as a standard, and two multiplexes that are now able to be used to provide presumptive identification of 12 species at one time.

In a specific embodiment, the first reference ladder comprises 143 bp DNA fragment from dog; 155 bp DNA fragment from goat; 226 bp DNA fragment from chicken and turkey; 273 bp DNA fragment from cow; 329 bp DNA fragment from sheep; 363 bp DNA fragment from cat; 394 bp DNA fragment from pig; and 436 bp DNA fragment from horse.

In a specific embodiment, the second reference ladder comprises 97 bp DNA fragment from cat; 196 bp DNA fragment from rabbit; 248 bp DNA fragment from human; 305 bp DNA fragment from dog; 314 bp DNA fragment from goat; and 328 bp DNA fragment from donkey.

In a specific embodiment, the first multiplex kit for use in PCR reaction to identify species from a sample comprises a universal forward primer of SEQ ID NO: 1, 17 or 18, and species-specific reverse primers of SEQ ID NOs: 2-9. In a specific embodiment, the second multiplex kit for use in PCR reaction to identify species from a sample comprises a universal forward primer of SEQ ID NO: 10, 19 or 20, and species-specific reverse primers of SEQ ID NOs: 11-16.

In certain embodiments, the method of the subject invention can be used as a presumptive test for forensic cases. If an unknown source of biological material is found at a crime scene, the kit and reference ladder can be used to identify the species of origin for that material. This allows for the investigators to proceed with the analyses based on the species identified (i.e., human, domestic animals/pets).

In forensics, reference ladders are critical to be able to bin the amplicons from the unknown sample to the correct amplicon in the reference ladder. No additional sequencing is needed to confirm. Most non-human DNA amplification kits on the market today do not have a reference ladder associated with the kit. Most kits that exist today only amplify 1-2 species at a time where the method of the subject invention can amplify 12 species at one time. No additional equipment is needed for a crime lab to use kit in any criminal cases. This can provide a presumptive test for animal abuse cases, illegal dog and cock fighting or identifying domestic animal/pet and human biological evidence associated with any crime scene.

In certain embodiments, the method of the subject invention can be used to identify, discriminate, or differentiate species in food fraud cases. Food fraud cases involve adulterated food products that can contain animal products not listed on the label. The reference ladder and multiplex kit allows for rapid identification of the contents. Other kits on the market identify only one or two species at a time are designed to be species specific. The subject invention provides an inexpensive, rapid analyses of adulterated food products that could contain any of the 12 species used in the kit that comprises a species reference ladder such that no other kit on the market has.

In certain embodiments, the kit of subject invention can be used as an educational kit for undergraduate molecular or forensic laboratories. While the kit is designed with fluorescently labeled primers in order to resolve amplicons using a genetic analyzer, the reference ladder and amplicons produced with the kit can also be resolved on an agarose gel typically used in a teaching laboratory so no need for expensive equipment.

The term “sample,” “forensic sample” or “forensic specimen” as used herein refers to various samples collected at crime scenes or large-scale disaster sites, and means a sample subjected to physical analysis, chemical analysis, molecular genetic analysis, and various other scientific analyses for forensic investigation. Forensic specimens may include various evidence at crime scenes, from which genes are detected, for example, saliva, blood, bloodstains, semen, vaginal fluid, vaginal secretions, vaginal wall discharges or mixtures thereof, hair, body hair, sweat, dead skin cells or mixtures thereof, clothes, underwear, handkerchiefs, masks, cigarette butts, cups, beverage bottles, cans, tissue paper, or the like. However, the forensic specimen of the present invention is not limited to the above substances and various samples collected at a crime scene, from which genomic DNA could be detected, may of course be used in the present invention.

As used herein, the singular forms “a,” “an,” and “the” are intended to include the plural forms as well, unless the context clearly indicates otherwise. Further, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof), such as “comprising,” “comprises,” and “comprise,” can be used interchangeably.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting of” or “consists essentially of” indicate that the claim encompasses embodiments containing the specified materials or steps and those that do not materially affect the basic and novel characteristic(s) of the claim. Use of the term “comprising” contemplates other embodiments that “consist of” or “consisting essentially of” the recited component(s).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed. In the context of compositions containing amounts of concentrations of ingredients where the term “about” is used, these values include a variation (error range) of 0-10% around the value (X±10%).

All percentages are by weight and all solvent mixture proportions are by volume unless otherwise noted. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

### Examples

Following are Examples which are offered by way of illustration and are not intended to limit the invention. Various modifications or changes in light thereof will be suggested to persons skilled in the art and are to be included within the spirit and purview of this application. Unless otherwise stated, these Examples utilized the methods, techniques, and materials as described in Materials and Methods above and known to those skilled in the art.

### Example 1—Multiplex 1 for Multi-Species Identification

Multiplex PCR kit is used for identification of domestic farm animals including dog, cat, sheep, goat, pig, chicken, turkey, horse, and cow. The PCR primers were created to target specific regions of the cytochrome b gene in mtDNA, where the universal forward is a universal primer for all species, and the individual reverse primers target the individual species to achieve distinct, unique amplicons (Table 1). Amplicon lengths for individual species are variable enough to give clear results. For instance, the fragment achieved when amplified chicken is 226 bp, and the fragment achieved when amplified horse is 436 bp. The same primer sets were used to create the species reference ladder that were in the multiplex reactions.

The universal forward primer (SEQ ID NO: 1) is labeled with a fluorescent dye such as FAM or NED. For example, fluorescent labeled universal forward primers of multiplex 1 include, for example, 5′-FAM-GACCTCCCAGCTCCATCAAACATCTCATCTTGATGAAA-3′ (SEQ ID NO: 17). Such fluorescently labeled primer, using FAM for example, can show up in the blue lane for capillary electrophoresis.

TABLE 1
Primers in multiplex 1

| | | | | | Frag |
|---|---|---|---|---|---|
| | | | F Tm | R Tm | Length |
| PRIMER | FORWARD | REVERSE | (C.) | (C.) | (bp) |
| Cat | 5′-GACCTCCCAGCTCCATCAAACATCTCATCTTGATGAAA- | 5′-CAGAAGGACATTTGGCCTCA-3′ | 64.2 | 55.1 | 363 |
| | 3′ (SEQ ID NO: 1) | (SEQ ID NO: 2) | | | |
| Dog | 5′-GACCTCCCAGCTCCATCAAACATCTCATCTTGATGAAA- | 5′-TGGGTGACTGATGAAAAAG 3′ | 64.2 | 50 | 143 |
| | 3′ (SEQ ID NO: 1) | (SEQ ID NO: 3) | | | |
| Goat | 5′-GACCTCCCAGCTCCATCAAACATCTCATCTTGATGAAA- | 5′-CTCGACAAATGTGAGTTACAGAG | 64.2 | 58.1 | 155 |
| | 3′ (SEQ ID NO: 1) | GGA-3′ (SEQ ID NO: 4) | | | |
| Chicken | 5′-GACCTCCCAGCTCCATCAAACATCTCATCTTGATGAAA- | 5′-AAGATACAGATGAAGAAGAATGA | 64.2 | 56.6 | 226 |
| | 3′ (SEQ ID NO: 1) | GGCG-3′ (SEQ ID NO: 5) | | | |
| Cow | 5′-GACCTCCCAGCTCCATCAAACATCTCATCTTGATGAAA- | 5′-CTAGAAAAGTGTAAGACCCGTAA | 64.2 | 53.2 | 273 |
| | 3′ (SEQ ID NO: 1) | TATAAG-3′ (SEQ ID NO: 6) | | | |
| Sheep | 5′-GACCTCCCAGCTCCATCAAACATCTCATCTTGATGAAA- | 5′-CTATGAATGCTGTGGCTATTGTCG | 64.2 | 59.3 | 329 |
| | 3′ (SEQ ID NO: 1) | CA-3′ (SEQ ID NO: 7) | | | |
| Pig | 5′-GACCTCCCAGCTCCATCAAACATCTCATCTTGATGAAA- | 5′-GCTGATAGTAGATTTGTGATGACC | 64.2 | 56.2 | 394 |
| | 3′ (SEQ ID NO: 1) | GTA-3′ (SEQ ID NO: 8) | | | |
| Horse | 5′-GACCTCCCAGCTCCATCAAACATCTCATCTTGATGAAA- | 5′-CTCAGATTCACTCGACGAGGGTAG | 64.2 | 58.8 | 436 |
| | 3′ (SEQ ID NO: 1) | TA-3′ (SEQ ID NO: 9) | | | |
| Uni- | 5′-FAM- | | | | |
| versal | GACCTCCCAGCTCCATCAAACATCTCATCTTGATGAAA -3′ | | | | |
| | (SEQ ID NO: 17) | | | | |

The universal forward and paired species-specific reverse primers were ran individually against all the species' DNA to test for cross-amplification. DNA is extracted from banked whole blood from the individual species, quantified and diluted to even concentrations. The universal forward and all species-specific primers were mixed in even concentrations and ran with mixed and non-mixed DNA to see how successful the amplification was. The resulting PCR product was run on the CE, and the peak height was used to adjust the various primer concentrations to the specific concentrations used in the multiplex. The thermocycler conditions were adjusted to ensure that each species had sufficient amplification, and all species were present. The reference ladder was constructed using individual PCR products from the singleplex rounds. The PCR product was cleaned using Exosap, quantified, diluted to even concentrations, and mixed together. The ladder was then viewed using CE, and adjusted to create even peak heights for each species in the ladder.

Multiplex 1 thermocycler conditions (25 cycles) are shown in Table 2. The ProFlex PCR System (ThermoFisher Scientific®, Waltham, MA) was used.

TABLE 2
Thermocycler Conditions

| | Degrees (° C.) | Time | |
|---|---|---|---|
| Step | | | |
| Step 1: Initial Denature | 95 | 15 | minutes |
| Step 2: Denature | 94 | 30 | seconds |
| Step 3: step down protocol: | | | |
| Anneal 1 | 59 | 90 | seconds |
| Anneal 2 | 55 | 90 | seconds |
| Anneal 3 | 53 | 90 | seconds |
| Anneal 4 | 50 | 90 | seconds |
| Step 4: Extend | 72 | 90 | seconds |
| Repeat Step 2-4 for 25 | | | |
| cycles | | | |
| Final Extension | 72 | 10 | minutes |
| Hold | 4 | 10 | minutes |

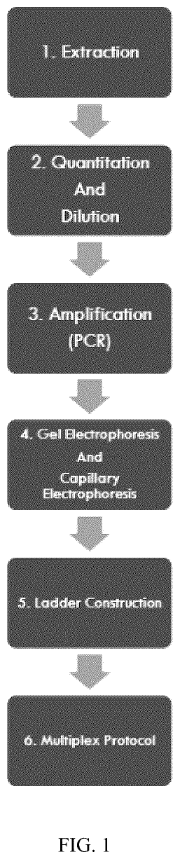

The traditional DNA workflow was used, including extraction, quantitation and dilution, amplification, and electrophoresis (FIG. 1). DNA was extracted from archived samples (e.g., whole blood) using a ZymoResearch® Quick DNA Microprep Plus Kit, using their extraction protocol for biological fluids and cells.

Quantitation on the extracted DNA samples was done using the Qubit 2.0 Fluorometer. This was done to standardize the concentration of DNA in the amplification stage. DNA concentration was read three times, averaged, and reported in ng/μL. After quantitating the extracted samples, they were diluted to a constant concentration of 1 ng/μL using the dilution C₁V₁=C₂V₂ equation and Hyclone water (nuclease free water).

Singleplex PCRs were performed to test cross amplification among the different species with their respective primers. Then, after ensuring no cross-amplification among the species, a multiplex PCR protocol was developed using a specific primer ratio.

Amplification was completed using Qiagen Multiplex PCR kit with a modified protocol. The mulitplex mix was used without the amplification additive called Q-solution, as it utilized chaperone molecules that result in non-target amplification due to ill-processed albumin. The PCR was done using 25 μL or 12.5 μL reactions that included the following: Qiagen multiplex mix, primer mix, hyclone water, and the template DNA. See the below table 3 for volumes and final concentrations.

TABLE 3
Multipex PCR mixtures

| | Per 1 RXN | | Per 1 RXN | Final |
|---|---|---|---|---|
| Reagent | (12.5 μL) | | (25 μL) | Concentration |
| 2X Qiagen Multiplex | 6.25 | μL | 12.5 μL | 1X |
| Mix | | | | |
| Primer mix (5 uM) | 1.25 | μL | 2.5 μL | 0.5 uM |
| Hyclone water | 4.0 | μL | 9.0 μL | — |
| DNA (1 ng/μL) | 1.0 | μL | 1.0 μL | 0.08 ng/μL (12.5 μL |
| (not included when | | | | RXN) |
| making master mix) | | | | 0.04 ng/μL (25 μL |
| | | | | RXN) |

The primer mix was designed to provide optimal amplification of each species, e.g., sufficient and equal amplification of each species. See table 4 for the primer mix specifications.

TABLE 4
Primer mixture details

| | Starting | Volume of Primer | | Final |
|---|---|---|---|---|
| Primer | Concentration | to add | | Concentration |
| Horse | 5 μM | 36 | μL | 0.82 μM |
| Cat | 5 μM | 15 | μL | 0.34 μM |
| Cow | 5 μM | 15 | μL | 0.34 μM |
| Chicken | 5 μM | 30 | μL | 0.68 μM |
| Goat | 5 μM | 30 | μL | 0.68 μM |
| Sheep | 5 μM | 9 | μL | 0.21 μM |
| Pig | 5 μM | 9 | μL | 0.31 μM |
| Dog | 5 μM | 30 | μL | 0.34 μM |
| Universal Forward | 5 μM | 45 | μL | 1.03 μM |
| (UFF) | | | | |
| | Total Volume | 219 | μL | |

Viewing PCR products was completed by using two forms of electrophoresis, gel electrophoresis and capillary electrophoresis. Gel electrophoresis was used to ensure the amplification was successful, to determine the rough size of the fragments, and to ensure that no cross amplification, nor non-target amplification occurred before using capillary electrophoresis.

Capillary electrophoresis (CE) was done to determine the exact fragment and peak intensity size using the ThermoFisher Scientific® SeqStudio on the fragment analysis setting using dye set (DS) 30 and GS-ROX500 as a size standard. The dye set can be changed to DS-32 once this project is combined with another to account for the compatibility of the different fluorescent dyes (NED and FAM). CE was set up using 9.25 μL of HiDi™ Formamide, 0.25 μL GS-ROX500, and 0.5 μL of PCR product, for an overall total volume of 10 μL.

The resulting electropherograms were analyzed using the Thermofisher Connect™ platform with microsatellite analysis (MSA).

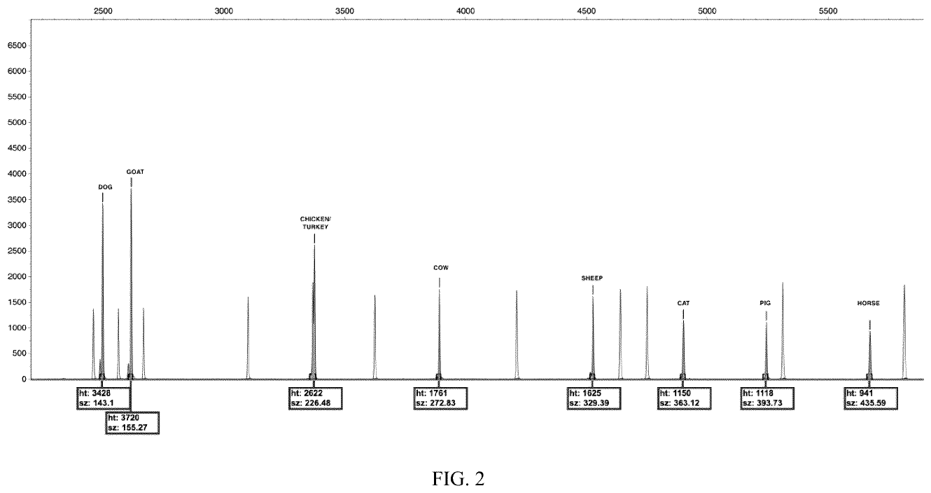

FIG. 2 shows the domestic animal reference ladder depicting from left to right: dog (143 bp), goat (155 bp), chicken and turkey (split peak 226 bp), cow (273 bp), sheep (329 bp), cat (363 bp), pig (394 bp) and horse (436 bp). The reference ladder was obtained using the universal forward primer of SEQ ID NO: 17 or 18 and species-specific reverse primers including SEQ ID NOs: 2-9.

The species reference ladder comprises fragments that have the sizes in Table 5.

TABLE 5
Species-specific fragments

| Species | Fragment Size (bp) |
|---|---|
| Dog | 143 |
| Goat | 155, 329 |
| Chicken | 226 |
| Turkey | 226 |
| Cow | 273 |
| Sheep | 329 |
| Cat | 363 |
| Pig | 394 |
| Horse | 436 |

These species are distinguished by length heterogeneity of the amplicons. Successful single-plex amplification of each species has been completed. The individual species' PCR products were column cleaned and then mixed to form the reference ladder. Then by amplifying any unknown evidence samples, the contributing species can quickly be identified when aligned with the known reference ladder using the genetic analyzer or gel electrophoresis.

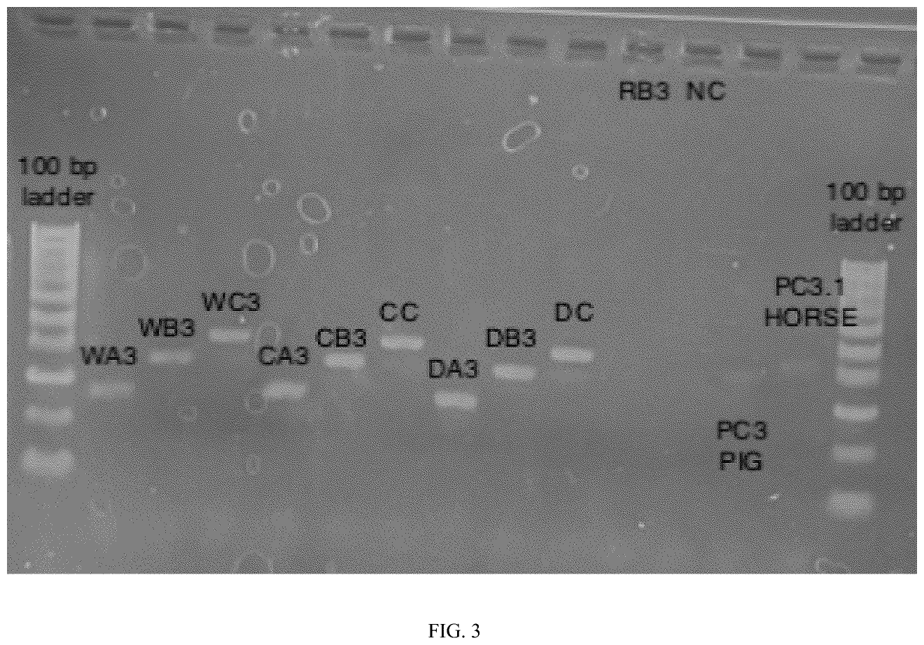

Next, to use the species-specific reference ladder for identifying an unknown species in a sample, unknown DNA samples were collected, processed and analyzed using gel electrophoresis. FIG. 3 shows the gel validation. The gel shows three unknows (A, B and C) in wood (W), cotton (C), and denim (D). The positive controls (PC) were faint, but present (pig and horse). This reaction was also viewed on CE. Also included in this gel is a reaction blank (BB), a negative control (NC), and a 100 bp standard sizing ladder.

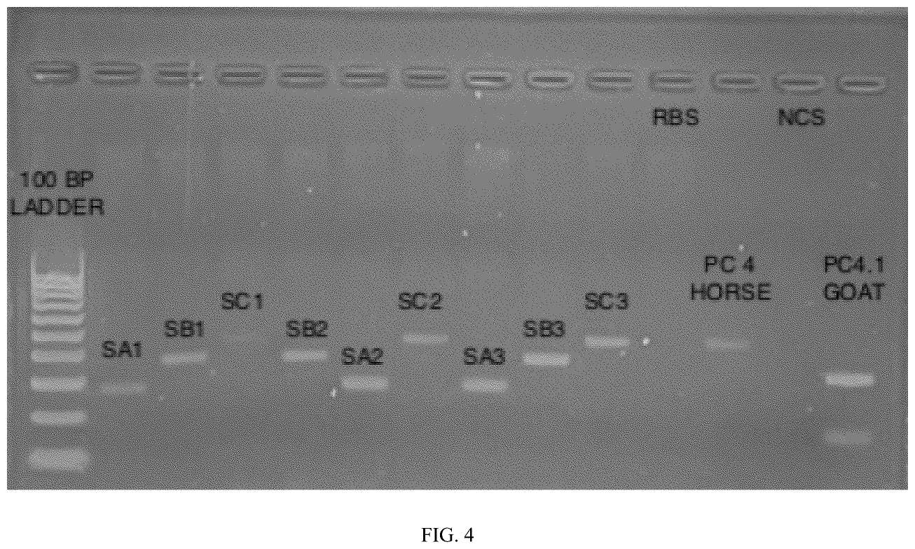

FIG. 4 shows the gel for the unknowns (A, B, and C) exposed to soil(S). the first set (1) was exposed to soil for approximately 1 month, while the other two (2 and 3) sets were exposed for roughly a day or so before extraction. This gel also includes a reaction blank (RBS), a negative control (NC), and two positive controls (PC4 and PC4.1), which were horse and goat, respectively. Goat is expected to have 2 bands under the multiplex conditions and is a distinct identifier for the species.

### Example 2—Mulitplex 2 for Multi-Species Identification

Multiplex PCR kit is used for identification of domestic and household pets including dog, cat, donkey, rabbit, goat, and human. The PCR primers were created to target specific regions of the cytochrome b gene in mtDNA, where the universal forward is a universal primer for all species, and the individual reverse primers target the individual species to achieve distinct, unique amplicons (Table 6). Amplicon lengths for individual species are variable enough to give clear results. The same primer sets were used to create the species reference ladder that were in the multiplex reactions.

The universal forward primer (SEQ ID NO: 10) is labeled with a fluorescent dye such as NED. Advantageously, NED fluoresce does not overlap as much with FAM as other fluorophores, such as HEX, do. For example, fluorescent labeled universal forward primers of multiplex 2 include, for example, 5′-NED-TGAGGACAAATATCATTYTGAGGRGC-3′ (SEQ ID NO: 20). Such fluorescently labeled primer, using NED for example, can show up in the black lane for capillary electrophoresis. Each of the species-specific reverse primers are unlabeled.

TABLE 6
Primers in multiplex 2

| | | | | | Frag |
|---|---|---|---|---|---|
| | | | F Tm | R Tm | Length |
| PRIMER | FORWARD | REVERSE | (C.) | (C.) | (bp) |
| Cat | 5′-TGAGGACAAATATCATTYTGAGGRGC-3′ | 5′-TTTCCCTCAGATYCATTCTACTAGTTCAG | 67.52 | 60.72 | 89 |
| | (SEQ ID NO: 10) | TC-3′ (SEQ ID NO: 11) | | | |
| Rabbit | 5′-TGAGGACAAATATCATTYTGAGGRGC-3′ | 5′-AAAGAGGAGGTGAATTAAGACTAAAGT-3′ | 67.52 | 66.98 | 192 |
| | (SEQ ID NO: 10) | (SEQ ID NO: 12) | | | |
| Human | 5′-TGAGGACAAATATCATTYTGAGGRGC-3′ | 5′-ATCGGAATGGGAGGTGATTCCTAGG-3′ | 67.52 | 71.23 | 246 |
| | (SEQ ID NO: 10) | (SEQ ID NO: 13) | | | |
| Dog | 5′-TGAGGACAAATATCATTYTGAGGRGC-3′ | 5′-GAGTAGGAGTAAGGCTCCTAGGATA-3′ | 67.52 | 60.94 | 303 |
| | (SEQ ID NO: 10) | (SEQ ID NO: 14) | | | |
| Donkey | 5′-TGAGGACAAATATCATTYTGAGGRGC-3′ | 5′-AATACTAGGGTTAGTAGGAGTAGGACTA-3′ | 67.52 | 58.15 | 327 |
| | (SEQ ID NO: 10) | (SEQ ID NO: 15) | | | |
| Goat | 5′-TGAGGACAAATATCATTYTGAGGRGC-3′ | 5′-TTAGAACAAGAATTAGTAGCATGGCG-3′ | 67.52 | 64.92 | 313 |
| | (SEQ ID NO: 10) | (SEQ ID NO: 16) | | | |
| Universal | 5′-NED-TGAGGACAAATATCATTYTGAGGR | | | | |
| | GC-3′ (SEQ ID NO: 20) | | | | |

Multiplex 2 thermocycler conditions (35 cycles) are shown in Table 7.

TABLE 7
Thermocycler Conditions

| | Degrees (° C.) | Time | |
|---|---|---|---|
| Step | | | |
| Initial activation | 95 | 15 | minutes |
| 3-Step cycling: | | | |
| Denaturation | 94 | 30 | seconds |
| Annealing | 60 | 90 | seconds |
| Extension | 72 | 90 | seconds |
| Repeat 35 cycles | | | |
| Final Extension | 72 | 10 | minutes |
| Hold | 4 | 10 | minutes |

The traditional DNA workflow was utilized, which consists of extraction, quantification, amplification, and capillary electrophoresis/gel electrophoresis. DNA was collected via blood samples in a K2/EDTA container from Lampire Biological Labs. DNA was extracted from blood using the ZymoResearch® manual extraction protocol for Non-Nucleated Biological Fluids & Cells. Quantification of DNA was calculated using the Qubit 2.0 Fluorometer that reports the concentration of DNA in ng/μL. The results were read in triplicate and then averaged.

Amplification was completed using a modified Qiagen® Standard Multiplex PCR protocol for individual reactions. The total reaction volume was cut in half from 50 μL to 25 μL, thus subsequently halving the amount of each component of the PCR. The universal forward and species-specific reverse primers were initially added individually at an equal concentration of 0.1 μM, however, when cross-reaction testing took place, primers were added in equal concentrations of 0.2 μM according to the Qiagen® protocol. For the multiplexed PCR reactions, the Qiagen® Multiplex Protocol for Microsatellite Loci will be used, again cutting the reaction volume in half from 50 μL to 25 μL. The ProFlex PCR System (ThermoFisher Scientific®, Waltham, MA) was used to execute the PCR cycle.

Before using capillary electrophoresis (CE), PCR products were examined on an agarose gel as a quality control to ensure amplification was successful without cross-amplification. Once this has been achieved, labeled PCR products will be analyzed using CE to determine the exact size of the amplicon.

Capillary electrophoresis will be accomplished using the ThermoFisher Scientific® SeqStudio on the fragment analysis setting using dye set 32 and GS-ROX500 to visualize results. Preparation of samples for capillary electrophoresis involved adding 9.25 μL of HiDi™ Formamide and 0.25 μL of size standard to 1 μL of PCR product. ThermoFisher Connect™ software using the microsatellite analysis app was used to visualize electropherograms.

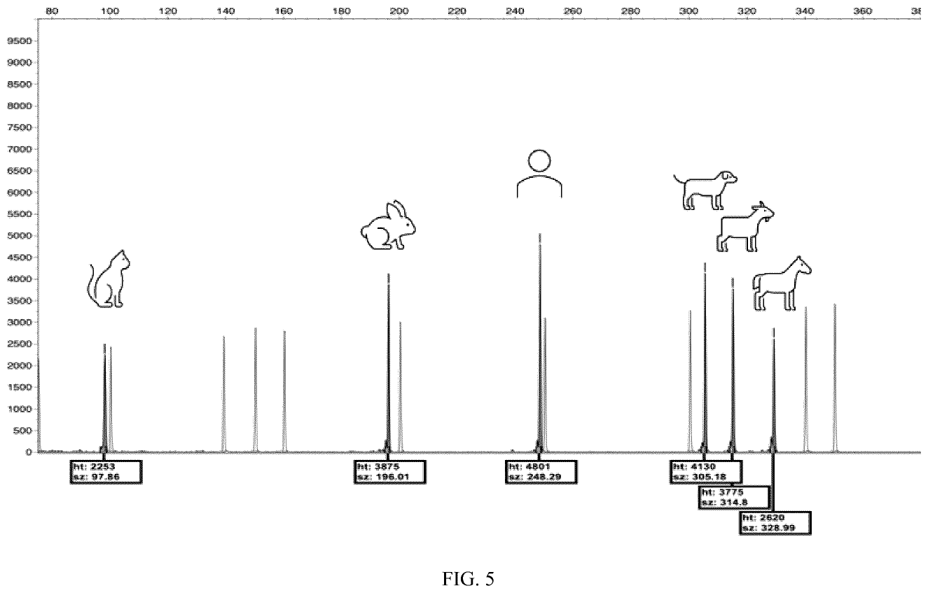

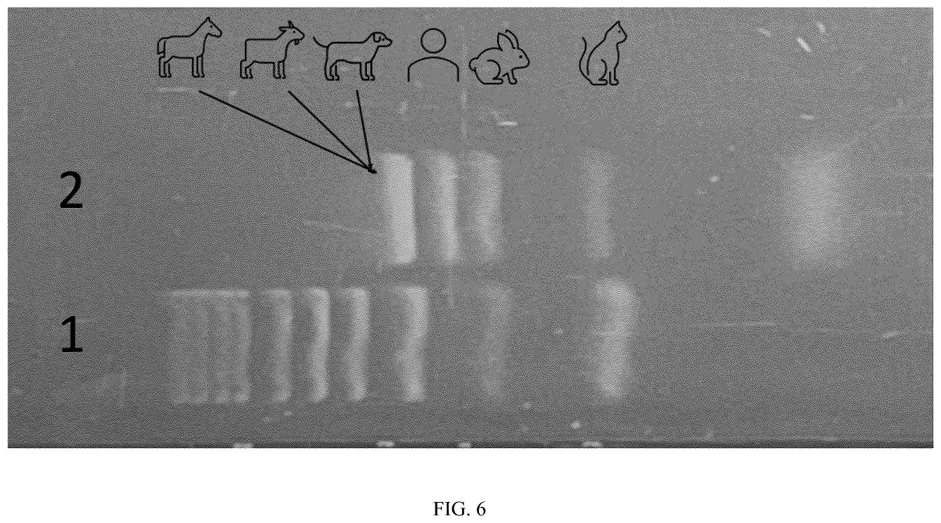

FIG. 5 shows electropherogram results for animal reference ladder made from combined fluorescently labeled (NED) PCR products, which was obtained using the universal forward primer of SEQ ID NO: 20 and species-specific reverse primers including SEQ ID NOs: 11-16. The ladder includes from left to right the following black peaks: cat (97 bp), rabbit (196 bp), human (248 bp), dog (305 bp), goat (314), and donkey (328 bp). The animal reference ladder was confirm using gel electrophoresis (FIG. 6).

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

What is claimed is:

1. A fluorescent multi-species reference ladder comprising two or more DNA fragments of cytochrome b (cyt-b) gene from two or more species, each of the two or more DNA fragments having a distinct length.

2. The fluorescent multi-species reference ladder of claim 1, the two or more species being selected from cat, cow, chicken, turkey, dog, donkey, goat, horse, pig, rabbit, sheep, and human.

3. The fluorescent multi-species reference ladder of claim 1, comprising two or more of:
about 97 bp DNA fragment from cat;
about 143 bp DNA fragment from dog;
about 155 bp DNA fragment from goat;
about 196 bp DNA fragment from rabbit;
about 226 bp DNA fragment from chicken and/or turkey;
about 248 bp DNA fragment from human;
about 273 bp DNA fragment from cow;
about 305 bp DNA fragment from dog;
about 314 bp DNA fragment from goat;
about 328 bp DNA fragment from donkey or about 329 bp DNA fragment from sheep;
about 363 bp DNA fragment from cat;
about 394 bp DNA fragment from pig; and
about 436 bp DNA fragment from horse.

4. The fluorescent multi-species reference ladder of claim 1, comprising:
about 143 bp DNA fragment from dog;
about 155 bp DNA fragment from goat;
about 226 bp DNA fragment from chicken and/or turkey;
about 273 bp DNA fragment from cow;
about 329 bp DNA fragment from sheep;
about 363 bp DNA fragment from cat;
about 394 bp DNA fragment from pig; and
about 436 bp DNA fragment from horse.

5. The fluorescent multi-species reference ladder of claim 1, comprising:
about 97 bp DNA fragment from cat;
about 196 bp DNA fragment from rabbit;
about 248 bp DNA fragment from human;
about 305 bp DNA fragment from dog;
about 314 bp DNA fragment from goat; and
about 328 bp DNA fragment from donkey.

6. The fluorescent multi-species reference ladder of claim 1, each of the two or more DNA fragments having a fluorescent label.

7. A kit comprising the fluorescent multi-species reference ladder of claim 1 and a mixture of primers, the primers comprising one or more fluorescent universal forward primers and two or more species-specific reverse primers targeting cyt-b gene in mtDNA.

8. The kit of claim 7, the one or more fluorescent universal forward primers comprising a sequence of SEQ ID NO: 17 or 18, and/or a sequence of SEQ ID NO: 19 or 20.

9. The kit of claim 7, the two or more species-specific reverse primers being selected from:
a sequence comprising SEQ ID NO: 2;
a sequence comprising SEQ ID NO: 3;
a sequence comprising SEQ ID NO: 4;
a sequence comprising SEQ ID NO: 5;
a sequence comprising SEQ ID NO: 6;
a sequence comprising SEQ ID NO: 7;
a sequence comprising SEQ ID NO: 8
a sequence comprising SEQ ID NO: 9;
a sequence comprising SEQ ID NO: 11;
a sequence comprising SEQ ID NO: 12;
a sequence comprising SEQ ID NO: 13;
a sequence comprising SEQ ID NO: 14;
a sequence comprising SEQ ID NO: 15; and
a sequence comprising SEQ ID NO: 16.

10. The kit of claim 7, the kit comprising species-specific reverse primers of SEQ ID NOs: 2-9.

11. The kit of claim 7, the kit comprising species-specific reverse primers of SEQ ID NOs: 11-16.

12. The kit of claim 7, the fluorescent universal forward primer and the two or more species-specific reverse primers being mixed at a ratio of from 1:0.4 to 1:4.

13. A method for species identification in a specimen, the method comprising preparing a DNA sample from the specimen, amplifying the DNA sample through multiplex PCR using the kit of claim 7, and determining the species in the specimen based on the result of the multiplex PCR.

14. The method of claim 13, the specimen being a forensic sample.

15. The method of claim 13, amplifying the DNA sample through multiplex PCR comprising mixing the primer mixture with the DNA sample; and subjecting said mixture of primer and DNA to multiplex PCR reactions to achieve Cyt-b gene amplicons.

16. The method of claim 15, the primer mixture comprising a fluorescent universal forward primer of SEQ ID NO: 17 or 18, and species-specific reverse primers of SEQ ID NOs: 2-9.

17. The method of claim 15, the primer mixture comprising a fluorescent universal forward primer of SEQ ID NO: 19 or 20, and species-specific reverse primers of SEQ ID NOs: 11-16.

18. The method of claim 13, determining the species in the specimen comprising comparing Cyt-b gene amplicons obtained from the multiplex PCR against the fluorescent multi-species reference ladder.

19. The method of claim 18, the fluorescent multi-species reference ladder comprising:
143 bp DNA fragment for dog;
155 bp DNA fragment for goat;
226 bp DNA fragment for chicken and/or turkey;
273 bp DNA fragment for cow;
329 bp DNA fragment for sheep;
363 bp DNA fragment for cat;
394 bp DNA fragment for pig; and
436 bp DNA fragment for horse.

20. The method of claim 18, the fluorescent multi-species reference ladder comprising:
97 bp DNA fragment for cat;
196 bp DNA fragment for rabbit;
248 bp DNA fragment for human;
305 bp DNA fragment for dog;
314 bp DNA fragment for goat; and
328 bp DNA fragment for donkey.
